(19) United States
(12) Patent Application Publication
(10) Pub. No.: US 2025/0250339 A1
(43) Pub. Date: Aug. 7, 2025

(54) ANTIBODIES TARGETING EGFR AND CD3 AND USES THEREOF

(71) Applicant: Janux Therapeutics, Inc., San Diego, CA (US)

(72) Inventors: David CAMPBELL, San Diego, CA (US); Thomas R. DIRAIMONDO, San Diego, CA (US)

(21) Appl. No.: 19/187,324

(22) Filed: Apr. 23, 2025

Related U.S. Application Data
(63) Continuation of application No. PCT/US23/78935, filed on Nov. 7, 2023.
(60) Provisional application No. 63/488,696, filed on Mar. 6, 2023.
(60) Provisional application No. 63/424,314, filed on Nov. 10, 2022.

(51) Int. Cl. C07K 16/28; A61P 35/00

(52) U.S. Cl. CPC C07K 16/2809; A61P 35/00; C07K 16/2863; C07K 2317/73; C07K 2317/92

(57) ABSTRACT

Provided herein are antibodies that selectively bind to EGFR and CD3, pharmaceutical compositions thereof, as well as methods of producing such antibodies.

### CROSS-REFERENCE

The present application is a continuation of International Patent Application No. PCT/US23/78935, filed Nov. 7, 2023, which claims the benefit of U.S. Provisional Application No. 63/424,314 filed on Nov. 10, 2022 and U.S. Provisional Application No. 63/488,696 filed on Mar. 6, 2023, each of which is incorporated herein by reference in its entirety.

### SEQUENCE LISTING

The instant application contains a Sequence Listing which has been submitted electronically in XML format and is hereby incorporated by reference in its entirety. Said XML copy, created on Oct. 20, 2023, is named 52426-749_301SL.xml and is 30,073 bytes in size.

### SUMMARY

Disclosed herein are isolated polypeptide complexes according to the formula:

A-L-B (Formula I)

wherein A comprises a single chain variable fragment (scFv) that binds to CD3, wherein the scFv comprises a scFv light chain variable domain and a scFv heavy chain variable domain comprising complementarity determining regions (CDRs): HC-CDR1, HC-CDR2, and HC-CDR3, wherein the HC-CDR1, the HC-CDR2, and the HC-CDR3 of the scFv heavy chain variable domain comprise HC-CDR1: SEQ ID NO: 1, HC-CDR2: SEQ ID NO: 2, and HC-CDR3: SEQ ID NO: 3; B comprises an antigen binding fragment (Fab) or Fab′ that binds to EGFR, wherein the Fab or Fab′ comprises a Fab light chain polypeptide comprising a Fab light chain variable domain and a Fab heavy chain polypeptide comprising a Fab heavy chain variable domain comprising complementarity determining regions (CDRs): HC-CDR1, HC-CDR2, and HC-CDR3, wherein the HC-CDR1, the HC-CDR2, and the HC-CDR3 of the Fab heavy chain variable domain comprise HC-CDR1: SEQ ID NO: 18, HC-CDR2: SEQ ID NO: 19, and HC-CDR3: SEQ ID NO: 20, and wherein the Fab heavy chain polypeptide comprises an N-glycosylation site having an amino acid sequence of N-X₁-X₂, wherein X₁ is any amino acid except P and X₂ is T or S; and L comprises a linker that connects A to B.

In some embodiments, the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise LC-CDR1: SEQ ID NO: 21, LC-CDR2: SEQ ID NO: 22, and LC-CDR3: SEQ ID NO: 23, and the CDRs comprise from 0-2 amino acid modifications in at least one of the LC-CDR1, LC-CDR2, or LC-CDR3. In some embodiments, the Fab light chain polypeptide comprises a mutation that eliminates pyroglutamic acid formation at an N-terminus of the Fab light chain polypeptide relative to a non-mutated form of the Fab light chain polypeptide. In some embodiments, the mutation is a Q to X₃ mutation at residue number 1 of the of the N-terminus of the Fab light chain polypeptide, and X₃ is an amino acid selected from the group consisting of: A, R, N, D, C, E, G, H, I, L, K, M, F, P, S, T, W, Y, or V. In some embodiments, X₃ is selected from D and E. In some embodiments, X₃ is D.

In some embodiments, the Fab light chain polypeptide comprises the amino acid sequence of SEQ ID NO: 24 or SEQ ID NO: 26. In some embodiments, the Fab light chain polypeptide comprises D at residue number 1 of the N-terminus of the Fab light chain polypeptide. In some embodiments, the Fab light chain polypeptide comprises the amino acid sequence of SEQ ID NO: 26. In some embodiments, the Fab light chain polypeptide comprises Q at residue number 1 of the N-terminus of the Fab light chain polypeptide. In some embodiments, the Fab light chain polypeptide comprises the amino acid sequence of SEQ ID NO: 24.

In some embodiments, X₂ of the N-glycosylation site is T. In some embodiments, X₁ of the N-glycosylation site is D. In some embodiments, the Fab heavy chain polypeptide that comprises the N-glycosylation site comprises an amino acid sequence QSNDTAIY (SEQ ID NO: 33). In some embodiments, the N of the N-glycosylation site is located at residue number 88 of the Fab heavy chain polypeptide. In some embodiments, the Fab heavy chain polypeptide comprises the amino acid sequence of SEQ ID NO: 25.

In some embodiments, the Fab light chain polypeptide comprises the amino acid sequence of SEQ ID NO: 24 and the Fab heavy chain polypeptide comprises the amino acid sequence of SEQ ID NO: 25. In some embodiments, the Fab light chain polypeptide consists of the amino acid sequence of SEQ ID NO: 24 and the Fab heavy chain polypeptide consists of the amino acid sequence of SEQ ID NO: 25. In some embodiments, the Fab light chain polypeptide comprises the amino acid sequence of SEQ ID NO: 26 and the Fab heavy chain polypeptide comprises the amino acid sequence of SEQ ID NO: 25. In some embodiments, the Fab light chain polypeptide consists of the amino acid sequence of SEQ ID NO: 26 and the Fab heavy chain polypeptide consists of the amino acid sequence of SEQ ID NO: 25.

In some embodiments, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise LC-CDR1: SEQ ID NO: 6, LC-CDR2: SEQ ID NO: 7, and LC-CDR3: SEQ ID NO: 8. In some embodiments, the scFv heavy chain variable domain comprises an amino acid sequence that has at least 80% sequence identity to the amino acid sequence of SEQ ID NO: 13. In some embodiments, the scFv heavy chain variable domain comprises an amino acid sequence of at least 100 consecutive amino acid residues of SEQ ID NO: 13. In some embodiments, the scFv heavy chain variable domain comprises the amino acid sequence of SEQ ID NO: 13. In some embodiments, the scFv light chain variable domain comprises an amino acid sequence that has at least 80% sequence identity to the amino acid sequence of SEQ ID NO: 12. In some embodiments, the scFv light chain variable domain comprises an amino acid sequence of at least 100 consecutive amino acid residues of SEQ ID NO: 12. In some embodiments, the scFv light chain variable domain comprises the amino acid sequence of SEQ ID NO: 12. In some embodiments, the scFv comprises an amino acid sequence that has at least 80% sequence identity to the amino acid sequence of SEQ ID NO: 14. In some embodiments, the scFv comprises an amino acid sequence of at least 200 consecutive amino acid residues of SEQ ID NO: 14. In some embodiments, the scFv comprises an amino acid sequence of at least 225 consecutive amino acid residues of SEQ ID NO: 14. In some embodiments, the scFv comprises the amino acid sequence of SEQ ID NO: 14.

In some embodiments, the linker connects the C-terminus of A to the N-terminus of B. In some embodiments, the linker connects the C-terminus of A to the N-terminus of the Fab light chain polypeptide. In some embodiments, the linker connects the C-terminus of A to the N-terminus of the Fab heavy chain polypeptide. In some embodiments, the linker connects the Fab heavy chain polypeptide to the C-terminus of the scFv light chain variable domain. In some embodiments, the linker connects the N-terminus of A to the C-terminus of B. In some embodiments, the linker connects the N-terminus of A to the C-terminus of the Fab heavy chain polypeptide. In some embodiments, the linker connects the N-terminus of A to the C-terminus of the Fab light chain polypeptide. In some embodiments, the linker is at least 5 amino acids in length. In some embodiments, the linker is no more than 10 amino acids in length. In some embodiments, the linker is no more than 30 amino acids in length. In some embodiments, the linker is at least 5 amino acids and no more than 30 amino acids in length. In some embodiments, the linker is 5 amino acids in length. In some embodiments, the linker comprises the amino acid sequence of SEQ ID NO: 28 (GGGGSGGGGSGGGGS), SEQ ID NO: 29 (GGGGS), or SEQ ID NO: 30 (GGGGSGGGS). In some embodiments, the linker comprises the amino acid sequence of SEQ ID NO: 29 (GGGGS).

In some embodiments, the linker connects the Fab heavy chain polypeptide to the C-terminus of the scFv light chain variable domain, and the Fab light chain polypeptide comprises an amino acid sequence that has at least 80% sequence identity to the amino acid sequence of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32. In some embodiments, the linker connects the Fab heavy chain polypeptide to the C-terminus of the scFv light chain variable domain, and the Fab light chain polypeptide comprises an amino acid sequence that has at least 90% sequence identity to the amino acid sequence of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32. In some embodiments, the linker connects the Fab heavy chain polypeptide to the C-terminus of the scFv light chain variable domain, and the Fab light chain polypeptide comprises an amino acid sequence of at least 200 consecutive amino acid residues of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32. In some embodiments, the linker connects the Fab heavy chain polypeptide to the C-terminus of the scFv light chain variable domain, and the Fab light chain polypeptide comprises an amino acid sequence according to SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

In some embodiments, the isolated polypeptide complex comprises an amino acid sequence with at least 90%, 95%, 96%, 97%, 98%, 99%, or 100% identity to SEQ ID NO: 24, and the amino acid sequence of SEQ ID NO: 32. In some embodiments, the isolated polypeptide complex comprises the amino acid sequences of SEQ ID NO: 24 and SEQ ID NO: 32. In some embodiments, the isolated polypeptide complex consists of the amino acid sequences of SEQ ID NO: 24 and SEQ ID NO: 32. In some embodiments, the isolated polypeptide complex comprises an amino acid sequence with at least 90%, 95%, 96%, 97%, 98%, 99%, or 100% identity to SEQ ID NO: 26, and the amino acid sequence of SEQ ID NO: 32. In some embodiments, the isolated polypeptide complex comprises the amino acid sequences of SEQ ID NO: 26 and SEQ ID NO: 32. In some embodiments, the isolated polypeptide complex consists of the amino acid sequences of SEQ ID NO: 26 and SEQ ID NO: 32.

Disclosed herein are isolated polypeptide complexes according to the formula:

A-L-B (Formula I)

wherein A comprises a single chain variable fragment (scFv) that binds to CD3, wherein the scFv comprises a scFv light chain variable domain and a scFv heavy chain variable domain; B comprises an antigen binding fragment (Fab) or Fab′ that binds to EGFR, wherein the Fab or Fab′ comprises a Fab light chain polypeptide comprising a Fab light chain variable domain and a Fab heavy chain polypeptide comprising a Fab heavy chain variable domain comprising complementarity determining regions (CDRs): HC-CDR1, HC-CDR2, and HC-CDR3, wherein the HC-CDR1, the HC-CDR2, and the HC-CDR3 of the Fab heavy chain variable domain comprise HC-CDR1: SEQ ID NO: 18, HC-CDR2: SEQ ID NO: 19, and HC-CDR3: SEQ ID NO: 20, and wherein the Fab heavy chain polypeptide comprises an N-glycosylation site having an amino acid sequence of N-X₁-X₂, wherein X₁ is any amino acid except P and X₂ is T or S; and L comprises a linker that connects A to B, wherein the linker connects the Fab heavy chain polypeptide to the scFv light chain variable domain.

In some embodiments, the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise LC-CDR1: SEQ ID NO: 21, LC-CDR2: SEQ ID NO: 22, and LC-CDR3: SEQ ID NO: 23, and wherein the CDRs comprise from 0-2 amino acid modifications in at least one of the LC-CDR1, LC-CDR2, or LC-CDR3. In some embodiments, the Fab light chain polypeptide comprises a mutation that eliminates pyroglutamic acid formation at an N-terminus of the Fab light chain polypeptide relative to a non-mutated form of the Fab light chain polypeptide. In some embodiments, the mutation is a Q to X₃ mutation at residue number 1 of the of the N-terminus of the Fab light chain polypeptide, and X₃ is an amino acid selected from the group consisting of: A, R, N, D, C, E, G, H, I, L, K, M, F, P, S, T, W, Y, or V. In some embodiments, X₃ is selected from D and E. In some embodiments, X₃ is D. In some embodiments, the Fab light chain polypeptide comprises D at residue number 1 of the N-terminus of the Fab light chain polypeptide. In some embodiments, the Fab light chain polypeptide comprises the amino acid sequence of SEQ ID NO: 24 or SEQ ID NO: 26. In some embodiments, the Fab light chain polypeptide comprises the amino acid sequence of SEQ ID NO: 26. In some embodiments, the Fab light chain polypeptide comprises Q at residue number 1 of the N-terminus of the Fab light chain polypeptide. In some embodiments, the Fab light chain polypeptide comprises the amino acid sequence of SEQ ID NO: 24.

In some embodiments, X₂ of the N-glycosylation site is T. In some embodiments, X₁ of the N-glycosylation site is D. In some embodiments, the Fab heavy chain polypeptide that comprises the N-glycosylation site comprises the amino acid sequence of QSNDTAIY (SEQ ID NO: 33). In some embodiments, the N of the N-glycosylation site is located at residue number 88 of the Fab heavy chain polypeptide. In some embodiments, the Fab heavy chain polypeptide comprises the amino acid sequence of SEQ ID NO: 25.

In some embodiments, the Fab light chain polypeptide comprises the amino acid sequence of SEQ ID NO: 24 and the Fab heavy chain polypeptide comprises the amino acid sequence of SEQ ID NO: 25. In some embodiments, the Fab light chain polypeptide consists of the amino acid sequence of SEQ ID NO: 24 and the Fab heavy chain polypeptide consists of the amino acid sequence of SEQ ID NO: 25. In some embodiments, the Fab light chain polypeptide comprises the amino acid sequence of SEQ ID NO: 26 and the Fab heavy chain polypeptide comprises the amino acid sequence of SEQ ID NO: 25. In some embodiments, the Fab light chain polypeptide consists of the amino acid sequence of SEQ ID NO: 26 and the Fab heavy chain polypeptide consists of the amino acid sequence of SEQ ID NO: 25.

In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise HC-CDR1: SEQ ID NO: 1, HC-CDR2: SEQ ID NO: 2, and HC-CDR3: SEQ ID NO: 3. In some embodiments, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise LC-CDR1: SEQ ID NO: 6, LC-CDR2: SEQ ID NO: 7, and LC-CDR3: SEQ ID NO: 8. In some embodiments, the scFv heavy chain variable domain comprises an amino acid sequence that has at least 80% sequence identity to the amino acid sequence of SEQ ID NO: 13. In some embodiments, the scFv heavy chain variable domain comprises an amino acid sequence of at least 100 consecutive amino acid residues of SEQ ID NO: 13. In some embodiments, the scFv heavy chain variable domain comprises the amino acid sequence of SEQ ID NO: 13. In some embodiments, the scFv light chain variable domain comprises an amino acid sequence that has at least 80% sequence identity to the amino acid sequence of SEQ ID NO: 12. In some embodiments, the scFv light chain variable domain comprises an amino acid sequence of at least 100 consecutive amino acid residues of SEQ ID NO: 12. In some embodiments, the scFv light chain variable domain comprises the amino acid sequence of SEQ ID NO: 12. In some embodiments, the scFv comprises an amino acid sequence that has at least 80% sequence identity to the amino acid sequence of SEQ ID NO: 14. In some embodiments, the scFv comprises an amino acid sequence of at least 200 consecutive amino acid residues of SEQ ID NO: 14. In some embodiments, the scFv comprises an amino acid sequence of at least 225 consecutive amino acid residues of SEQ ID NO: 14. In some embodiments, the scFv comprises the amino acid sequence of SEQ ID NO: 14.

In some embodiments, the linker connects the C-terminus of A to the N-terminus of B. In some embodiments, the linker connects the C-terminus of A to the N-terminus of the Fab heavy chain polypeptide. In some embodiments, the linker connects the Fab heavy chain polypeptide to the C-terminus of the scFv light chain variable domain. In some embodiments, the linker connects the N-terminus of the Fab heavy chain polypeptide to the C-terminus of the scFv light chain variable domain. In some embodiments, the linker connects the N-terminus of A to the C-terminus of B. In some embodiments, the linker is at least 5 amino acids in length. In some embodiments, the linker is no more than 10 amino acids in length. In some embodiments, the linker is no more than 30 amino acids in length. In some embodiments, the linker is at least 5 amino acids and no more than 30 amino acids in length. In some embodiments, the linker is 5 amino acids in length. In some embodiments, the linker comprises the amino acid sequence of SEQ ID NO: 28 (GGGGSGGGGSGGGGS), SEQ ID NO: 29 (GGGGS), or SEQ ID NO: 30 (GGGGSGGGS). In some embodiments, the linker comprises the amino acid sequence of SEQ ID NO: 29 (GGGGS).

In some embodiments, the linker connects the Fab heavy chain polypeptide to the C-terminus of the scFv light chain variable domain, and the Fab light chain polypeptide comprises an amino acid sequence that has at least 80% sequence identity to the amino acid sequence of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32. In some embodiments, the linker connects the Fab heavy chain polypeptide to the C-terminus of the scFv light chain variable domain, and the Fab light chain polypeptide comprises an amino acid sequence that has at least 90% sequence identity to the amino acid sequence of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32. In some embodiments, the linker connects the Fab heavy chain polypeptide to the C-terminus of the scFv light chain variable domain, and the Fab light chain polypeptide comprises an amino acid sequence of at least 200 consecutive amino acid residues of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32. In some embodiments, the linker connects the Fab heavy chain polypeptide to the C-terminus of the scFv light chain variable domain, and the Fab light chain polypeptide comprises an amino acid sequence according to SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

In some embodiments, the isolated polypeptide complex comprises an amino acid sequence with at least 90%, 95%, 96%, 97%, 98%, 99%, or 100% identity to SEQ ID NO: 24, and the amino acid sequence of SEQ ID NO: 32. In some embodiments, the isolated polypeptide complex comprises amino acid sequences of SEQ ID NO: 24 and SEQ ID NO: 32. In some embodiments, the isolated polypeptide complex consists of amino acid sequences of SEQ ID NO: 24 and SEQ ID NO: 32. In some embodiments, the isolated polypeptide complex comprises an amino acid sequence with at least 90%, 95%, 96%, 97%, 98%, 99%, or 100% identity to SEQ ID NO: 26, and the amino acid sequence of SEQ ID NO: 32. In some embodiments, the isolated polypeptide complex comprises amino acid sequences of SEQ ID NO: 26 and SEQ ID NO: 32. In some embodiments, the isolated polypeptide complex consists of amino acid sequences of SEQ ID NO: 26 and SEQ ID NO: 32.

Additionally disclosed herein are pharmaceutical compositions comprising (i) the isolated polypeptide complex according to any embodiment herein, and (ii) a pharmaceutically acceptable excipient.

Additionally disclosed herein are isolated recombinant nucleic acid molecules encoding an isolated polypeptide complex according to any embodiment herein.

Further disclosed herein are methods of treating cancer in a subject comprising administering to the subject the isolated polypeptide complex of any embodiment herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an exemplary configuration of a polypeptide complex that selectively binds to EGFR and CD3;

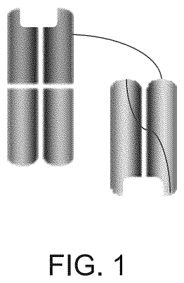

FIG. 2 illustrates Ab-1, Ab-2, and Ab-3 binding to EGFR measured by ELISA;

FIG. 3 illustrates Ab-1, Ab-2, and Ab-3 binding to CD3 measured by ELISA; and

FIG. 4 illustrates killing of HCT116 tumor cell killing mediated by Ab-1, Ab-2, and Ab-3 in the presence of CD8+ T cells.

### DETAILED DESCRIPTION

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “antibody” is used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like.

The term “complementarity determining region” or “CDR” is a segment of the variable region of an antibody that is complementary in structure to the epitope to which the antibody binds and is more variable than the rest of the variable region. Accordingly, a CDR is sometimes referred to as hypervariable region. A variable region comprises three CDRs. CDR peptides can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2:106 (1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles and Applications, Birch et al., (eds.), pages 137-185 (Wiley-Liss, Inc. 1995).

The term “Fab” refers to a protein that contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme); and Whitelegg N R and Rees A R, “WAM: an improved algorithm for modelling antibodies on the WEB,” Protein Eng. 2000 December; 13 (12): 819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

### Isolated Polypeptide Complexes

Multispecific antibodies combine the benefits of different binding specificities derived from two or more antibodies into a single composition. Multispecific antibodies for redirecting T cells to cancers have shown promise in both pre-clinical and clinical studies. This approach relies on binding of one antigen interacting portion of the antibody to a tumor-associated antigen or marker, while a second antigen interacting portion can bind to an effector cell antigen on a T cell, such as CD3, which then triggers cytotoxic activity.

One such tumor-associated antigen is EGFR. Epidermal growth factor receptor (EGFR), also known as ERBB1, receptor tyrosine-protein kinase ErbB-1, or proto-oncogene C ErbB-1 is a transmembrane glycoprotein that is a member of the protein kinase superfamily. EGFR is a cell surface protein that binds to epidermal growth factor, thus inducing receptor dimerization and tyrosine autophosphorylation leading to cell proliferation. Amplification and mutations in EGFR have been shown to be driving events in many cancer types.

Disclosed herein are antibodies that selectively bind to EGFR and CD3, in which the anti-EGFR domain is in a Fab or Fab′ antibody format that is linked to a single-chain variable fragment (scFv) that binds to CD3. The bispecific antibody format of a Fab or Fab′ linked to a scFv provides efficacy and safety advantages over other bispecific antibody formats.

Disclosed herein are isolated polypeptide complexes according to the formula:

A-L-B (Formula I)

wherein A comprises a single chain variable fragment (scFv) that binds to CD3, wherein the scFv comprises a scFv light chain variable domain and a scFv heavy chain variable domain comprising complementarity determining regions (CDRs): HC-CDR1, HC-CDR2, and HC-CDR3, wherein the HC-CDR1, the HC-CDR2, and the HC-CDR3 of the scFv heavy chain variable domain comprise HC-CDR1: SEQ ID NO: 1, HC-CDR2: SEQ ID NO: 2, and HC-CDR3: SEQ ID NO: 3 (see Table 1); B comprises an antigen binding fragment (Fab) or Fab′ that binds to EGFR, wherein the Fab or Fab′ comprises a Fab light chain polypeptide comprising a Fab light chain variable domain and a Fab heavy chain polypeptide comprising a Fab heavy chain variable domain comprising complementarity determining regions (CDRs): HC-CDR1, HC-CDR2, and HC-CDR3, wherein the HC-CDR1, the HC-CDR2, and the HC-CDR3 of the Fab heavy chain variable domain comprise HC-CDR1: SEQ ID NO: 18, HC-CDR2: SEQ ID NO: 19, and HC-CDR3: SEQ ID NO: 20 (see Table 4), and the Fab heavy chain polypeptide comprises an N-glycosylation site having an amino acid sequence of N-X₁-X₂, wherein X₁ is any amino acid except P and X₂ is T or S; and L comprises a linker that connects A to B.

TABLE 1
anti-CD3 scFv heavy chain variable domain
complementarity determining regions (CDR)s
(as based on the IMGT CDR numbering system).

| | Amino Acid Sequence | SEQ |
|---|---|---|
| Construct Description | (N to C) | ID NO: |
| CD3 1: CDR-H1 | GFTFNKYA | 1 |
| CD3 1: CDR-H2 | IRSKYNNYAT | 2 |
| CD3 1: CDR-H3 | VRHGNFGNSYISYWAY | 3 |
| CD3 2: CDR-H1 | GFTFNTYA | 4 |
| CD3 2: CDR-H2 | IRSKYNNYAT | 2 |
| CD3 2: CDR-H3 | VRHGNFGNSYVSWFAY | 5 |

TABLE 2
anti-CD3 scFv light chain variable domain
complementarity determining regions (CDR)s
(as based on the IMGT CDR numbering system).

| | Amino Acid Sequence | SEQ |
|---|---|---|
| Construct Description | (N to C) | ID NO: |
| CD3 1: CDR-L1 | TGAVTSGNY | 6 |
| CD3 1: CDR-L2 | GTK | 7 |
| CD3 1: CDR-L3 | VLWYSNRWV | 8 |
| CD3 2: CDR-L1 | TGAVTTSNY | 9 |
| CD3 2: CDR-L2 | GTN | 10 |
| CD3 2: CDR-L3 | ALWYSNLWV | 11 |

TABLE 3
anti-CD3 scFv light chain variable domain,
heavy chain variable domain sequences,
and full length sequence

| Construct | Amino Acid Sequence | SEQ |
|---|---|---|
| Description | (N to C) | ID NO: |
| CD3 1 scFv: LC | QTVVTQEPSLTVSPGGTVTLTCGSSTGA | 12 |
| | VTSGNYPNWVQQKPGQAPRGLIGGTKF | |
| | LAPGTPARFSGSLLGGKAALTLSGVQPE | |
| | DEAEYYCVLWYSNRWVFGGGTKLTVL | |
| CD3 1 scFv: HC | EVQLVESGGGLVQPGGSLKLSCAASGFT | 13 |
| | FNKYAMNWVRQAPGKGLEWVARIRSK | |
| | YNNYATYYADSVKDRFTISRDDSKNTAY | |
| | LQMNNLKTEDTAVYYCVRHGNFGNSYI | |
| | SYWAYWGQGTLVTVSS | |
| CD3 1 scFv | EVQLVESGGGLVQPGGSLKLSCAASGFT | 14 |
| | FNKYAMNWVRQAPGKGLEWVARIRSK | |
| | YNNYATYYADSVKDRFTISRDDSKNTAY | |
| | LQMNNLKTEDTAVYYCVRHGNFGNSYI | |
| | SYWAYWGQGTLVTVSSGGGGSGGGGS | |
| | GGGGSQTVVTQEPSLTVSPGGTVTLTCG | |
| | SSTGAVTSGNYPNWVQQKPGQAPRGLI | |
| | GGTKFLAPGTPARFSGSLLGGKAALTLS | |
| | GVQPEDEAEYYCVLWYSNRWVFGGGT | |
| | KLTVL | |
| CD3 2 scFv VL | QTVVTQEPSLTVSPGGTVTLTCRSSTGA | 15 |
| | VTTSNYANWVQQKPGQAPRGLIGGTNK | |
| | RAPGTPARFSGSLLGGKAALTLSGVQPE | |
| | DEAEYYCALWYSNLWVFGGGTKLTVL | |
| CD3 2 scFv VH | EVQLVESGGGLVQPGGSLKLSCAASGFT | 16 |
| | FNTYAMNWVRQAPGKGLEWVARIRSK | |
| | YNNYATYYADSVKDRFTISRDDSKNTAY | |
| | LQMNNLKTEDTAVYYCVRHGNFGNSY | |
| | VSWFAYWGQGTLVTVSS | |
| CD3 2 scFv | QTVVTQEPSLTVSPGGTVTLTCRSSTGA | 17 |
| | VTTSNYANWVQQKPGQAPRGLIGGTNK | |
| | RAPGTPARFSGSLLGGKAALTLSGVQPE | |
| | DEAEYYCALWYSNLWVFGGGTKLTVL | |
| | GGGGSGGGGSGGGGSEVQLVESGGGLV | |
| | QPGGSLKLSCAASGFTFNTYAMNWVRQ | |
| | APGKGLEWVARIRSKYNNYATYYADSV | |
| | KDRFTISRDDSKNTAYLQMNNLKTEDTA | |
| | VYYCVRHGNFGNSYVSWFAYWGQGTL | |
| | VTVSS | |

TABLE 4
anti-EGFR Fab heavy chain variable domain
complementarity determining regions(CDR)s
(as based on the IMGT CDR numbering system).

| Construct | Amino Acid Sequence | SEQ |
|---|---|---|
| Description | (N to C) | ID NO: |
| EGFR 1: CDR-H1 | GFSLTNYG | 18 |
| EGFR 1: CDR-H2 | IWSGGNT | 19 |
| EGFR 1: CDR-H3 | ARALTYYDYEFAY | 20 |

TABLE 5
anti-EGFR Fab light chain variable domain
complementarity determining regions (CDR)s
(as based on the IMGT CDR numbering system).

| Construct | Amino Acid Sequence | SEQ |
|---|---|---|
| Description | (N to C) | ID NO: |
| EGFR 1: CDR-L1 | QSIGTN | 21 |
| EGFR 1: CDR-L2 | YAS | 22 |
| EGFR 1: CDR-L3 | QQNNNWPTT | 23 |

Antigen Binding Fragment (Fab or Fab′) that Binds to EGFR

In some embodiments, the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise LC-CDR1: SEQ ID NO: 21, LC-CDR2: SEQ ID NO: 22, and LC-CDR3: SEQ ID NO: 23 (see Table 5), and wherein the CDRs comprise from 0-2 amino acid modifications in at least one of the LC-CDR1, LC-CDR2, or LC-CDR3. In some embodiments, the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise LC-CDR1: SEQ ID NO: 21, LC-CDR2: SEQ ID NO: 22, and LC-CDR3: SEQ ID NO: 23.

In some embodiments, the Fab light chain polypeptide comprises a mutation that eliminates pyroglutamic acid formation at an N-terminus of the Fab light chain polypeptide relative to a non-mutated form of the Fab light chain polypeptide. In some embodiments, the mutation is a Q to X₃ mutation at residue number 1 of the of the N-terminus of the Fab light chain polypeptide, and wherein X₃ is an amino acid selected from the group consisting of: A, R, N, D, C, E, G, H, I, L, K, M, F, P, S, T, W, Y, or V. In some embodiments, X₃ is A. In some embodiments, X₃ is R. In some embodiments, X₃ is N. In some embodiments, X₃ is C. In some embodiments, X₃ is E. In some embodiments, X₃ is G. In some embodiments, X₃ is H. In some embodiments, X₃ is I. In some embodiments, X₃ is L. In some embodiments, X₃ is K. In some embodiments, X₃ is M. In some embodiments, X₃ is F. In some embodiments, X₃ is P. In some embodiments, X₃ is S. In some embodiments, X₃ is T. In some embodiments, X₃ is W. In some embodiments, X₃ is Y. In some embodiments, X₃ is V. In some embodiments, X₃ is selected from D and E. In some embodiments, X₃ is D.

In some embodiments, the Fab light chain polypeptide comprises the amino acid sequence of SEQ ID NO: 24 or SEQ ID NO: 26 (see Table 6). In some embodiments, the Fab light chain polypeptide comprises D at residue number 1 of the N-terminus of the Fab light chain polypeptide. In some embodiments, the Fab light chain polypeptide comprises the amino acid sequence of SEQ ID NO: 26. In some embodiments, the Fab light chain polypeptide comprises Q at residue number 1 of the N-terminus of the Fab light chain polypeptide. In some embodiments, the Fab light chain polypeptide comprises the amino acid sequence of SEQ ID NO: 24.

TABLE 6
anti-EGFR Fab light chain polypeptide and Fab
heavy chain polypeptide sequences

| | | SEQ |
|---|---|---|
| Construct | Amino Acid Sequence | ID |
| Description | (N to C) | NO: |
| EGFR 1 Fab | QILLTQSPVILSVSPGERVSFSCRASQSIGTNI | 24 |
| LC | HWYQQRINGSPRLLIKYASESISGIPSRFSGS | |
| | GSGTDFTLSINSVESEDIADYYCQQNNNWP | |
| | TTFGAGTKLELKRTVAAPSVFIFPPSDEQLK | |
| | SGTASVVCLLNNFYPREAKVQWKVDNALQ | |
| | SGNSQESVTEQDSKDSTYSLSSTLTLSKADY | |
| | EKHKVYACEVTHQGLSSPVTKSFNRGEC | |
| EGFR 1 Fab | QVQLKQSGPGLVQPSQSLSITCTVSGFSLTN | 25 |
| HC | YGVHWVRQSPGKGLEWLGVIWSGGNTDY | |
| | NTPFTSRLSINKDNSKSQVFFKMNSLQSNDT | |
| | AIYYCARALTYYDYEFAYWGQGTLVTVSA | |
| | ASTKGPSVFPLAPSSKSTSGGTAALGCLVKD | |
| | YFPEPVTVSWNSGALTSGVHTFPAVLQSSG | |
| | LYSLSSVVTVPSSSLGTQTYICNVNHKPSNT | |
| | KVDKKVEPKSC | |
| EGFR 2 Fab | DILLTQSPVILSVSPGERVSFSCRASQSIGTNI | 26 |
| LC | HWYQQRTNGSPRLLIKYASESISGIPSRFSGS | |
| | GSGTDFTLSINSVESEDIADYYCQQNNNWP | |
| | TTFGAGTKLELKRTVAAPSVFIFPPSDEQLK | |
| | SGTASVVCLLNNFYPREAKVQWKVDNALQ | |
| | SGNSQESVTEQDSKDSTYSLSSTLTLSKADY | |
| | EKHKVYACEVTHQGLSSPVTKSFNRGEC | |
| EGFR 2 Fab | QVQLKQSGPGLVQPSQSLSITCTVSGFSLTN | 27 |
| HC | YGVHWVRQSPGKGLEWLGVIWSGGNTDY | |
| | NTPFTSRLSINKDNSKSQVFFKMNSLQSQDT | |
| | AIYYCARALTYYDYEFAYWGQGTLVTVSA | |
| | ASTKGPSVFPLAPSSKSTSGGTAALGCLVKD | |
| | YFPEPVTVSWNSGALTSGVHTFPAVLQSSG | |
| | LYSLSSVVTVPSSSLGTQTYICNVNHKPSNT | |
| | KVDKKVEPKSC | |

In some embodiments, X₂ of the N-glycosylation site is T. In some embodiments, X₁ of the N-glycosylation site is D. In some embodiments, X₁ of the N-glycosylation site is A. In some embodiments, X₁ of the N-glycosylation site is R. In some embodiments, X₁ of the N-glycosylation site is N. In some embodiments, X₁ of the N-glycosylation site is C. In some embodiments, X₁ of the N-glycosylation site is E. In some embodiments, X₁ of the N-glycosylation site is Q. In some embodiments, X₁ of the N-glycosylation site is G. In some embodiments, X₁ of the N-glycosylation site is H. In some embodiments, X₁ of the N-glycosylation site is I. In some embodiments, X₁ of the N-glycosylation site is L. In some embodiments, X₁ of the N-glycosylation site is K. In some embodiments, X₁ of the N-glycosylation site is M. In some embodiments, X₁ of the N-glycosylation site is F. In some embodiments, X₁ of the N-glycosylation site is S. In some embodiments, X₁ of the N-glycosylation site is T. In some embodiments, X₁ of the N-glycosylation site is W. In some embodiments, X₁ of the N-glycosylation site is Y. In some embodiments, X₁ of the N-glycosylation site is V. In some embodiments, the N-glycosylation site has the amino acid sequence N-D-T. In some embodiments, the Fab heavy chain polypeptide that comprises the N-glycosylation site comprises an amino acid sequence QSNDTAIY (SEQ ID NO: 33). In some embodiments, the N of the N-glycosylation site is located at residue number 88 of the Fab heavy chain polypeptide.

In some embodiments, the Fab heavy chain polypeptide comprises the amino acid sequence of SEQ ID NO: 25 (see Table 6).

In some embodiments, the Fab light chain polypeptide comprises an amino acid sequence with at least 80% identity to SEQ ID NO: 24, and the Fab heavy chain polypeptide comprises an amino acid sequence with at least 80% identity to SEQ ID NO: 25. In some embodiments, the Fab light chain polypeptide comprises an amino acid sequence with at least 90% identity to SEQ ID NO: 24, and the Fab heavy chain polypeptide comprises an amino acid sequence with at least 90% identity to SEQ ID NO: 25. In some embodiments, the Fab light chain polypeptide comprises an amino acid sequence with at least 95% identity to SEQ ID NO: 24, and the Fab heavy chain polypeptide comprises an amino acid sequence with at least 95% identity to SEQ ID NO: 25. In some embodiments, the Fab light chain polypeptide comprises an amino acid sequence of at least 200 consecutive amino acid residues of SEQ ID NO: 24, and the Fab heavy chain polypeptide comprises an amino acid sequence of at least 200 consecutive amino acid residues of SEQ ID NO: 25. In some embodiments, the Fab light chain polypeptide comprises the amino acid sequence of SEQ ID NO: 24 and the Fab heavy chain polypeptide comprises the amino acid sequence of SEQ ID NO: 25. In some embodiments, the Fab light chain polypeptide consists of the amino acid sequence of SEQ ID NO: 24 and the Fab heavy chain polypeptide consists of the amino acid sequence of SEQ ID NO: 25.

In some embodiments, the Fab light chain polypeptide comprises an amino acid sequence with at least 80% identity to SEQ ID NO: 26, and the Fab heavy chain polypeptide comprises an amino acid sequence with at least 80% identity to SEQ ID NO: 25. In some embodiments, the Fab light chain polypeptide comprises an amino acid sequence with at least 90% identity to SEQ ID NO: 26, and the Fab heavy chain polypeptide comprises an amino acid sequence with at least 90% identity to SEQ ID NO: 25. In some embodiments, the Fab light chain polypeptide comprises an amino acid sequence with at least 95% identity to SEQ ID NO: 26, and the Fab heavy chain polypeptide comprises an amino acid sequence with at least 95% identity to SEQ ID NO: 25. In some embodiments, the Fab light chain polypeptide comprises an amino acid sequence of at least 200 consecutive amino acid residues of SEQ ID NO: 26, and the Fab heavy chain polypeptide comprises an amino acid sequence of at least 200 consecutive amino acid residues of SEQ ID NO: 25. In some embodiments, the Fab light chain polypeptide comprises the amino acid sequence of SEQ ID NO: 26 and the Fab heavy chain polypeptide comprises the amino acid sequence of SEQ ID NO: 25. In some embodiments, the Fab light chain polypeptide consists of the amino acid sequence of SEQ ID NO: 26 and the Fab heavy chain polypeptide consists of the amino acid sequence of SEQ ID NO: 25.

Single Chain Variable Fragment (scFv) that Binds to CD3

In some embodiments, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise LC-CDR1: SEQ ID NO: 6, LC-CDR2: SEQ ID NO: 7, and LC-CDR3: SEQ ID NO: 8, and the CDRs comprise from 0-2 amino acid modifications in at least one of the LC-CDR1, LC-CDR2, or LC-CDR3 (see Table 2). In some embodiments, the LC-CDR1, the LC-CDR2, and the LC-CDR3 of the scFv light chain variable domain comprise LC-CDR1: SEQ ID NO: 6, LC-CDR2: SEQ ID NO: 7, and LC-CDR3: SEQ ID NO: 8.

In some embodiments, the scFv heavy chain variable domain comprises an amino acid sequence that has at least 80% sequence identity to the amino acid sequence of SEQ ID NO: 13 (see Table 3). In some embodiments, the scFv heavy chain variable domain comprises an amino acid sequence of at least 100 consecutive amino acid residues of SEQ ID NO: 13. In some embodiments, the scFv heavy chain variable domain comprises the amino acid sequence of SEQ ID NO: 13. In some embodiments, the scFv heavy chain variable domain consists of the amino acid sequence of SEQ ID NO: 13.

In some embodiments, the scFv light chain variable domain comprises an amino acid sequence that has at least 80% sequence identity to the amino acid sequence of SEQ ID NO: 12 (see Table 3). In some embodiments, the scFv light chain variable domain comprises an amino acid sequence of at least 100 consecutive amino acid residues of SEQ ID NO: 12. In some embodiments, the scFv light chain variable domain comprises the amino acid sequence of SEQ ID NO: 12. In some embodiments, the scFv light chain variable domain consists of the amino acid sequence of SEQ ID NO: 12.

In some embodiments, the scFv comprises an amino acid sequence that has at least 80% sequence identity to the amino acid sequence of SEQ ID NO: 14 (see Table 3). In some embodiments, the scFv comprises an amino acid sequence of at least 200 consecutive amino acid residues of SEQ ID NO: 14. In some embodiments, the scFv comprises an amino acid sequence of at least 225 consecutive amino acid residues of SEQ ID NO: 14. In some embodiments, the scFv comprises the amino acid sequence of SEQ ID NO: 14. In some embodiments, the scFv consists of the amino acid sequence of SEQ ID NO: 14.

### Linker and Connections

In some embodiments, the linker connects the C-terminus of A to the N-terminus of B. In some embodiments, the linker connects the C-terminus of A to the N-terminus of the Fab light chain polypeptide. In some embodiments, the linker connects the C-terminus of A to the N-terminus of the Fab heavy chain polypeptide. In some embodiments, the linker connects the Fab heavy chain polypeptide to the C-terminus of the scFv light chain variable domain. In some embodiments, the linker connects the N-terminus of the Fab heavy chain polypeptide to the C-terminus of the scFv light chain variable domain.

In some embodiments, the linker connects the N-terminus of A to the C-terminus of B. In some embodiments, the linker connects the N-terminus of A to the C-terminus of the Fab heavy chain polypeptide. In some embodiments, the linker connects the N-terminus of A to the C-terminus of the Fab light chain polypeptide.

In some embodiments, the linker is at least 5 amino acids in length. In some embodiments, the linker is no more than 10 amino acids in length. In some embodiments, the linker is no more than 30 amino acids in length. In some embodiments, the linker is at least 5 amino acids and no more than 30 amino acids in length. In some embodiments, the linker is 5 amino acids in length. In some embodiments, the linker comprises the amino acid sequence of SEQ ID NO: 28 (GGGGSGGGGSGGGGS), SEQ ID NO: 29 (GGGGS), or SEQ ID NO: 30 (GGGGSGGGS) (see Table 7). In some embodiments, the linker comprises the amino acid sequence of SEQ ID NO: 29 (GGGGS).

TABLE 7
Linker sequences

| Construct | Amino Acid Sequence | SEQ |
|---|---|---|
| Description | (N to C) | ID NO: |
| Linker | GGGGSGGGGSGGGGS | 28 |
| Linker | GGGGS | 29 |
| Linker | GGGGSGGGS | 30 |

In some embodiments, the linker connects the Fab heavy chain polypeptide to the C-terminus of the scFv light chain variable domain, and the Fab light chain polypeptide comprises an amino acid sequence that has at least 80% sequence identity to the amino acid sequence of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

In some embodiments, the linker connects the Fab heavy chain polypeptide to the C-terminus of the scFv light chain variable domain, and the Fab light chain polypeptide comprises an amino acid sequence that has at least 90% sequence identity to the amino acid sequence of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

In some embodiments, the linker connects the Fab heavy chain polypeptide to the C-terminus of the scFv light chain variable domain, and the Fab light chain polypeptide comprises an amino acid sequence of at least 200 consecutive amino acid residues of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

In some embodiments, the linker connects the Fab heavy chain polypeptide to the C-terminus of the scFv light chain variable domain, and the Fab light chain polypeptide comprises an amino acid sequence according to SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

### Polypeptide Complex Sequences

In some embodiments, the isolated polypeptide complex comprises an amino acid sequence with at least 90%, 92%, 95%, 96%, 97%, 98%, 99%, or 100% identity to SEQ ID NO: 24, and an amino acid sequence with at least 90%, 95%, 96%, 97%, 98%, 99%, or 100% identity to SEQ ID NO: 32. In some embodiments, the isolated polypeptide complex comprises an amino acid sequence with at least 90% identity to SEQ ID NO: 24, and an amino acid sequence with at least 90% identity to SEQ ID NO: 32. In some embodiments, the isolated polypeptide complex comprises an amino acid sequence with at least 92% identity to SEQ ID NO: 24, and an amino acid sequence with at least 92% identity to SEQ ID NO: 32. In some embodiments, the isolated polypeptide complex comprises an amino acid sequence with at least 95% identity to SEQ ID NO: 24, and an amino acid sequence with at least 95% identity to SEQ ID NO: 32. In some embodiments, the isolated polypeptide complex comprises an amino acid sequence with at least 96% identity to SEQ ID NO: 24, and an amino acid sequence with at least 96% identity to SEQ ID NO: 32. In some embodiments, the isolated polypeptide complex comprises an amino acid sequence with at least 97% identity to SEQ ID NO: 24, and an amino acid sequence with at least 97% identity to SEQ ID NO: 32. In some embodiments, the isolated polypeptide complex comprises an amino acid sequence with at least 98% identity to SEQ ID NO: 24, and an amino acid sequence with at least 98% sequence identity to SEQ ID NO: 32. In some embodiments, the isolated polypeptide complex comprises an amino acid sequence with at least 99% identity to SEQ ID NO: 24, and an amino acid sequence with at least 99% identity to SEQ ID NO: 32. In some embodiments, the isolated polypeptide complex comprises the amino acid sequences of SEQ ID NO: 24 and SEQ ID NO: 32. In some embodiments, the isolated polypeptide complex consists of the amino acid sequences of SEQ ID NO: 24 and SEQ ID NO: 32.

In some embodiments, the isolated polypeptide complex comprises an amino acid sequence with at least 90%, 92%, 95%, 96%, 97%, 98%, 99%, or 100% identity to SEQ ID NO: 26, and an amino acid sequence at least 90%, 92%, 95%, 96%, 97%, 98%, 99%, or 100% identity to SEQ ID NO: 32. In some embodiments, the isolated polypeptide complex comprises an amino acid sequence with at least 90% identity to SEQ ID NO: 26, and an amino acid sequence with at least 90% sequence identity to SEQ ID NO: 32. In some embodiments, the isolated polypeptide complex comprises an amino acid sequence with at least 92% identity to SEQ ID NO: 26, and an amino acid sequence with at least 92% sequence identity to SEQ ID NO: 32. In some embodiments, the isolated polypeptide complex comprises an amino acid sequence with at least 95% identity to SEQ ID NO: 26, and an amino acid sequence with at least 95% sequence identity to SEQ ID NO: 32. In some embodiments, the isolated polypeptide complex comprises an amino acid sequence with at least 97% identity to SEQ ID NO: 26, and an amino acid sequence with at least 97% identity to SEQ ID NO: 32. In some embodiments, the isolated polypeptide complex comprises an amino acid sequence with at least 98% identity to SEQ ID NO: 26, and an amino acid sequence with at least 98% sequence identity to SEQ ID NO: 32. In some embodiments, the isolated polypeptide complex comprises an amino acid sequence with at least 99% identity to SEQ ID NO: 26, and an amino acid sequence with at least 99% sequence identity to SEQ ID NO: 32. In some embodiments, the isolated polypeptide complex comprises the amino acid sequences of SEQ ID NO: 26 and SEQ ID NO: 32. In some embodiments, the isolated polypeptide complex consists of the amino acid sequences of SEQ ID NO: 26 and SEQ ID NO: 32.

In some embodiments, the isolated polypeptide complex comprises a modified amino acid or non-natural amino acid, or a modified non-natural amino acid, or a combination thereof. In some embodiments, the modified amino acid or a modified non-natural amino acid comprises a post-translational modification. In some embodiments the isolated polypeptide complex comprises a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to the isolated polypeptide complex including the peptide backbone, the amino acid side chains, and the terminus.

Disclosed herein are isolated polypeptide complexes according to the formula:

A-L-B (Formula I)

wherein A comprises a single chain variable fragment (scFv) that binds to CD3, wherein the scFv comprises a scFv light chain variable domain and a scFv heavy chain variable domain; B comprises an antigen binding fragment (Fab) or Fab′ that binds to EGFR, wherein the Fab or Fab′ comprises a Fab light chain polypeptide comprising a Fab light chain variable domain and a Fab heavy chain polypeptide comprising a Fab heavy chain variable domain comprising complementarity determining regions (CDRs): HC-CDR1, HC-CDR2, and HC-CDR3, wherein the HC-CDR1, the HC-CDR2, and the HC-CDR3 of the Fab heavy chain variable domain comprise HC-CDR1: SEQ ID NO: 18, HC-CDR2: SEQ ID NO: 19, and HC-CDR3: SEQ ID NO: 20, and wherein the Fab heavy chain polypeptide comprises an N-glycosylation site having an amino acid sequence of N-X₁-X₂, wherein X₁ is any amino acid except P and X₂ is T or S; and L comprises a linker that connects A to B, wherein the linker connects the Fab heavy chain polypeptide to the scFv light chain variable domain.
Antigen Binding Fragment (Fab or Fab′) that Binds to EGFR

In some embodiments, the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise LC-CDR1: SEQ ID NO: 21, LC-CDR2: SEQ ID NO: 22, and LC-CDR3: SEQ ID NO: 23, and the CDRs comprise from 0-2 amino acid modifications in at least one of the LC-CDR1, LC-CDR2, or LC-CDR3. In some embodiments, the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise LC-CDR1: SEQ ID NO: 21, LC-CDR2: SEQ ID NO: 22, and LC-CDR3: SEQ ID NO: 23.

In some embodiments, the Fab light chain polypeptide comprises a mutation that eliminates pyroglutamic acid formation at an N-terminus of the Fab light chain polypeptide relative to a non-mutated form of the Fab light chain polypeptide. In some embodiments, the mutation is a Q to X₃ mutation at residue number 1 of the of the N-terminus of the Fab light chain polypeptide, and wherein X₃ is an amino acid selected from the group consisting of: A, R, N, D, C, E, G, H, I, L, K, M, F, P, S, T, W, Y, or V. In some embodiments, X₃ is A. In some embodiments, X₃ is R. In some embodiments, X₃ is N. In some embodiments, X₃ is C. In some embodiments, X₃ is E. In some embodiments, X₃ is G. In some embodiments, X₃ is H. In some embodiments, X₃ is I. In some embodiments, X₃ is L. In some embodiments, X₃ is K. In some embodiments, X₃ is M. In some embodiments, X₃ is F. In some embodiments, X₃ is P. In some embodiments, X₃ is S. In some embodiments, X₃ is T. In some embodiments, X₃ is W. In some embodiments, X₃ is Y. In some embodiments, X₃ is V. In some embodiments, X₃ is selected from D and E. In some embodiments, X₃ is D.

In some embodiments, the Fab light chain polypeptide comprises the amino acid sequence of SEQ ID NO: 24 or SEQ ID NO: 26. In some embodiments, the Fab light chain polypeptide comprises D at residue number 1 of the N-terminus of the Fab light chain polypeptide. In some embodiments, the Fab light chain polypeptide comprises the amino acid sequence of SEQ ID NO: 26. In some embodiments, the Fab light chain polypeptide comprises Q at residue number 1 of the N-terminus of the Fab light chain polypeptide. In some embodiments, the Fab light chain polypeptide comprises the amino acid sequence of SEQ ID NO: 24.

In some embodiments, X₂ of the N-glycosylation site is T. In some embodiments, X₁ of the N-glycosylation site is D. In some embodiments, X₁ of the N-glycosylation site is A. In some embodiments, X₁ of the N-glycosylation site is R. In some embodiments, X₁ of the N-glycosylation site is N. In some embodiments, X₁ of the N-glycosylation site is C. In some embodiments, X₁ of the N-glycosylation site is E. In some embodiments, X₁ of the N-glycosylation site is Q. In some embodiments, X₁ of the N-glycosylation site is G. In some embodiments, X₁ of the N-glycosylation site is H. In some embodiments, X₁ of the N-glycosylation site is I. In some embodiments, X₁ of the N-glycosylation site is L. In some embodiments, X₁ of the N-glycosylation site is K. In some embodiments, X₁ of the N-glycosylation site is M. In some embodiments, X₁ of the N-glycosylation site is F. In some embodiments, X₁ of the N-glycosylation site is S. In some embodiments, X₁ of the N-glycosylation site is T. In some embodiments, X₁ of the N-glycosylation site is W. In some embodiments, X₁ of the N-glycosylation site is Y. In some embodiments, X₁ of the N-glycosylation site is V. In some embodiments, the N-glycosylation site has the amino acid sequence N-D-T. In some embodiments, the Fab heavy chain polypeptide that comprises the N-glycosylation site comprises an amino acid sequence QSNDTAIY (SEQ ID NO: 33). In some embodiments, the N of the N-glycosylation site is located at residue number 88 of the Fab heavy chain polypeptide.

In some embodiments, the Fab heavy chain polypeptide comprises the amino acid sequence of SEQ ID NO: 25.

In some embodiments, the Fab light chain polypeptide comprises an amino acid sequence with at least 80% identity to SEQ ID NO: 24, and the Fab heavy chain polypeptide comprises an amino acid sequence with at least 80% identity to SEQ ID NO: 25. In some embodiments, the Fab light chain polypeptide comprises an amino acid sequence with at least 90% identity to SEQ ID NO: 24, and the Fab heavy chain polypeptide comprises an amino acid sequence with at least 90% identity to SEQ ID NO: 25. In some embodiments, the Fab light chain polypeptide comprises an amino acid sequence with at least 95% identity to SEQ ID NO: 24, and the Fab heavy chain polypeptide comprises an amino acid sequence with at least 95% identity to SEQ ID NO: 25. In some embodiments, the Fab light chain polypeptide comprises an amino acid sequence of at least 200 consecutive amino acid residues of SEQ ID NO: 24, and the Fab heavy chain polypeptide comprises an amino acid sequence of at least 200 consecutive amino acid residues of SEQ ID NO: 25. In some embodiments, the Fab light chain polypeptide comprises the amino acid sequence of SEQ ID NO: 24 and the Fab heavy chain polypeptide comprises the amino acid sequence of SEQ ID NO: 25. In some embodiments, the Fab light chain polypeptide consists of the amino acid sequence of SEQ ID NO: 24 and the Fab heavy chain polypeptide consists of the amino acid sequence of SEQ ID NO: 25.

In some embodiments, the Fab light chain polypeptide comprises an amino acid sequence with at least 80% identity to SEQ ID NO: 26, and the Fab heavy chain polypeptide comprises an amino acid sequence with at least 80% identity to SEQ ID NO: 25. In some embodiments, the Fab light chain polypeptide comprises an amino acid sequence with at least 90% identity to SEQ ID NO: 26, and the Fab heavy chain polypeptide comprises an amino acid sequence with at least 90% identity to SEQ ID NO: 25. In some embodiments, the Fab light chain polypeptide comprises an amino acid sequence with at least 95% identity to SEQ ID NO: 26, and the Fab heavy chain polypeptide comprises an amino acid sequence with at least 95% identity to SEQ ID NO: 25. In some embodiments, the Fab light chain polypeptide comprises an amino acid sequence of at least 200 consecutive amino acid residues of SEQ ID NO: 26, and the Fab heavy chain polypeptide comprises an amino acid sequence of at least 200 consecutive amino acid residues of SEQ ID NO: 25. In some embodiments, the Fab light chain polypeptide comprises the amino acid sequence of SEQ ID NO: 26 and the Fab heavy chain polypeptide comprises the amino acid sequence of SEQ ID NO: 25. In some embodiments, the Fab light chain polypeptide consists of the amino acid sequence of SEQ ID NO: 26 and the Fab heavy chain polypeptide consists of the amino acid sequence of SEQ ID NO: 25.

Single Chain Variable Fragment (scFv) that Binds to CD3

In some embodiments, the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise HC-CDR1: SEQ ID NO: 1, HC-CDR2: SEQ ID NO: 2, and HC-CDR3: SEQ ID NO: 3, and the CDRs comprise from 0-2 amino acid modifications in at least one of HC-CDR1, HC-CDR2, or HC-CDR3. In some embodiments, the HC-CDR1, the HC-CDR2, and the HC-CDR3 of the scFv heavy chain variable domain comprise HC-CDR1: SEQ ID NO: 1, HC-CDR2: SEQ ID NO: 2, and HC-CDR3: SEQ ID NO: 3.

In some embodiments, the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise LC-CDR1: SEQ ID NO: 6, LC-CDR2: SEQ ID NO: 7, and LC-CDR3: SEQ ID NO: 8, and the CDRs comprise from 0-2 amino acid modifications in at least one of the LC-CDR1, LC-CDR2, or LC-CDR3. In some embodiments, the LC-CDR1, the LC-CDR2, and the LC-CDR3 of the scFv light chain variable domain comprise LC-CDR1: SEQ ID NO: 6, LC-CDR2: SEQ ID NO: 7, and LC-CDR3: SEQ ID NO: 8.

In some embodiments, the scFv heavy chain variable domain comprises an amino acid sequence that has at least 80% sequence identity to the amino acid sequence of SEQ ID NO: 13. In some embodiments, the scFv heavy chain variable domain comprises an amino acid sequence of at least 100 consecutive amino acid residues of SEQ ID NO: 13. In some embodiments, the scFv heavy chain variable domain comprises the amino acid sequence of SEQ ID NO: 13. In some embodiments, the scFv heavy chain variable domain consists of the amino acid sequence of SEQ ID NO: 13.

In some embodiments, the scFv light chain variable domain comprises an amino acid sequence that has at least 80% sequence identity to the amino acid sequence of SEQ ID NO: 12. In some embodiments, the scFv light chain variable domain comprises an amino acid sequence of at least 100 consecutive amino acid residues of SEQ ID NO: 12. In some embodiments, the scFv light chain variable domain comprises the amino acid sequence of SEQ ID NO: 12. In some embodiments, the scFv light chain variable domain consists of the amino acid sequence of SEQ ID NO: 12.

In some embodiments, the scFv comprises an amino acid sequence that has at least 80% sequence identity to the amino acid sequence of SEQ ID NO: 14. In some embodiments, the scFv comprises an amino acid sequence of at least 200 consecutive amino acid residues of SEQ ID NO: 14. In some embodiments, the scFv comprises an amino acid sequence of at least 225 consecutive amino acid residues of SEQ ID NO: 14. In some embodiments, the scFv comprises the amino acid sequence of SEQ ID NO: 14. In some embodiments, the scFv consists of the amino acid sequence of SEQ ID NO: 14.

### Linker and Connections

In some embodiments, the linker connects the C-terminus of A to the N-terminus of B. In some embodiments, the linker connects the C-terminus of A to the N-terminus of the Fab heavy chain polypeptide. In some embodiments, the linker connects the Fab heavy chain polypeptide to the C-terminus of the scFv light chain variable domain. In some embodiments, the linker connects the N-terminus of the Fab heavy chain polypeptide to the C-terminus of the scFv light chain variable domain.

In some embodiments, the linker connects the N-terminus of A to the C-terminus of B. In some embodiments, the linker connects the N-terminus of A to the C-terminus of the Fab heavy chain polypeptide. In some embodiments, the linker connects the Fab heavy chain polypeptide to the N-terminus of the scFv light chain variable domain. In some embodiments, the linker connects the C-terminus of the Fab heavy chain polypeptide to the N-terminus of the scFv light chain variable domain.

In some embodiments, the linker is at least 5 amino acids in length. In some embodiments, the linker is no more than 10 amino acids in length. In some embodiments, the linker is no more than 30 amino acids in length. In some embodiments, the linker is at least 5 amino acids and no more than 30 amino acids in length. In some embodiments, the linker is 5 amino acids in length. In some embodiments, the linker comprises the amino acid sequence of SEQ ID NO: 28 (GGGGSGGGGSGGGGS), SEQ ID NO: 29 (GGGGS), or SEQ ID NO: 30 (GGGGSGGGS). In some embodiments, the linker comprises the amino acid sequence of SEQ ID NO: 29 (GGGGS).

In some embodiments, the linker connects the Fab heavy chain polypeptide to the C-terminus of the scFv light chain variable domain, and the Fab light chain polypeptide comprises an amino acid sequence that has at least 80% sequence identity to the amino acid sequence of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

In some embodiments, the linker connects the Fab heavy chain polypeptide to the C-terminus of the scFv light chain variable domain, and the Fab light chain polypeptide comprises an amino acid sequence that has at least 90% sequence identity to the amino acid sequence of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

In some embodiments, the linker connects the Fab heavy chain polypeptide to the C-terminus of the scFv light chain variable domain, and the Fab light chain polypeptide comprises an amino acid sequence of at least 200 consecutive amino acid residues of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

In some embodiments, the linker connects the Fab heavy chain polypeptide to the C-terminus of the scFv light chain variable domain, and the Fab light chain polypeptide comprises an amino acid sequence according to SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

### Polypeptide Complex Sequences

In some embodiments, the isolated polypeptide complex comprises an amino acid sequence with at least 90%, 92%, 95%, 96%, 97%, 98%, 99%, or 100% identity to SEQ ID NO: 24, and an amino acid sequence with at least 90%, 92%, 95%, 96%, 97%, 98%, 99%, or 100% identity to SEQ ID NO: 32. In some embodiments, the isolated polypeptide complex comprises an amino acid sequence with at least 90% identity to SEQ ID NO: 24, and an amino acid sequence with at least 90% identity to SEQ ID NO: 32. In some embodiments, the isolated polypeptide complex comprises an amino acid sequence with at least 92% identity to SEQ ID NO: 24, and an amino acid sequence with at least 92% identity to SEQ ID NO: 32. In some embodiments, the isolated polypeptide complex comprises an amino acid sequence with at least 95% identity to SEQ ID NO: 24, and an amino acid sequence with at least 95% identity to SEQ ID NO: 32. In some embodiments, the isolated polypeptide complex comprises an amino acid sequence with at least 96% identity to SEQ ID NO: 24, and an amino acid sequence with at least 96% identity to SEQ ID NO: 32. In some embodiments, the isolated polypeptide complex comprises an amino acid sequence with at least 97% identity to SEQ ID NO: 24, and an amino acid sequence with at least 97% identity to SEQ ID NO: 32. In some embodiments, the isolated polypeptide complex comprises an amino acid sequence with at least 98% identity to SEQ ID NO: 24, and an amino acid sequence with at least 98% identity to SEQ ID NO: 32. In some embodiments, the isolated polypeptide complex comprises an amino acid sequence with at least 99% identity to SEQ ID NO: 24, and an amino acid sequence with at least 99% identity to SEQ ID NO: 32. In some embodiments, the isolated polypeptide complex comprises the amino acid sequences of SEQ ID NO: 24 and SEQ ID NO: 32. In some embodiments, the isolated polypeptide complex consists of the amino acid sequences of SEQ ID NO: 24 and SEQ ID NO: 32.

In some embodiments, the isolated polypeptide complex comprises an amino acid sequence with at least 90%, 92%, 95%, 96%, 97%, 98%, 99%, or 100% identity to SEQ ID NO: 26, and an amino acid sequence 90%, 92%, 95%, 96%, 97%, 98%, 99%, or 100% identity to SEQ ID NO: 32. In some embodiments, the isolated polypeptide complex comprises an amino acid sequence with at least 90% identity to SEQ ID NO: 26, and an amino acid sequence with at least 90% identity to SEQ ID NO: 32. In some embodiments, the isolated polypeptide complex comprises an amino acid sequence with at least 92% identity to SEQ ID NO: 26, and an amino acid sequence with at least 92% identity to SEQ ID NO: 32. In some embodiments, the isolated polypeptide complex comprises an amino acid sequence with at least 95% identity to SEQ ID NO: 26, and an amino acid sequence with at least 95% identity to SEQ ID NO: 32. In some embodiments, the isolated polypeptide complex comprises an amino acid sequence with at least 97% identity to SEQ ID NO: 26, and an amino acid sequence with at least 97% identity to SEQ ID NO: 32. In some embodiments, the isolated polypeptide complex comprises an amino acid sequence with at least 98% identity to SEQ ID NO: 26, and an amino acid sequence with at least 98% identity to SEQ ID NO: 32. In some embodiments, the isolated polypeptide complex comprises an amino acid sequence with at least 99% identity to SEQ ID NO: 26, and an amino acid sequence with at least 99% identity to SEQ ID NO: 32. In some embodiments, the isolated polypeptide complex comprises the amino acid sequences of SEQ ID NO: 26 and SEQ ID NO: 32. In some embodiments, the isolated polypeptide complex consists of the amino acid sequences of SEQ ID NO: 26 and SEQ ID NO: 32.

In some embodiments, the isolated polypeptide complex comprises a modified amino acid or non-natural amino acid, or a modified non-natural amino acid, or a combination thereof. In some embodiments, the modified amino acid or a modified non-natural amino acid comprises a post-translational modification. In some embodiments the isolated polypeptide complex comprises a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to the isolated polypeptide complex including the peptide backbone, the amino acid side chains, and the terminus.

### Pharmaceutical Compositions

Disclosed herein, in some embodiments, are pharmaceutical compositions comprising: (i) an isolated polypeptide or polypeptide complex of any embodiment disclosed herein; and (ii) a pharmaceutically acceptable excipient.

Disclosed herein, in some embodiments, are pharmaceutical compositions comprising an isolated polypeptide complex according to the following formula:

A-L-B (Formula I)

wherein A comprises a single chain variable fragment (scFv) that binds to CD3, wherein the scFv comprises a scFv light chain variable domain and a scFv heavy chain variable domain comprising complementarity determining regions (CDRs): HC-CDR1, HC-CDR2, and HC-CDR3, wherein the HC-CDR1, the HC-CDR2, and the HC-CDR3 of the scFv heavy chain variable domain comprise HC-CDR1: SEQ ID NO: 1, HC-CDR2: SEQ ID NO: 2, and HC-CDR3: SEQ ID NO: 3; B comprises an antigen binding fragment (Fab) or Fab′ that binds to EGFR, wherein the Fab or Fab′ comprises a Fab light chain polypeptide comprising a Fab light chain variable domain and a Fab heavy chain polypeptide comprising a Fab heavy chain variable domain comprising complementarity determining regions (CDRs): HC-CDR1, HC-CDR2, and HC-CDR3, wherein the HC-CDR1, the HC-CDR2, and the HC-CDR3 of the Fab heavy chain variable domain comprise HC-CDR1: SEQ ID NO: 18, HC-CDR2: SEQ ID NO: 19, and HC-CDR3: SEQ ID NO: 20, and wherein the Fab heavy chain polypeptide comprises an N-glycosylation site having an amino acid sequence of N-X₁-X₂, wherein X₁ is any amino acid except P and X₂ is T or S; and L comprises a linker that connects A to B.

Disclosed herein, in some embodiments, are pharmaceutical compositions comprising an isolated polypeptide complex according to the following formula:

A-L-B (Formula I)

wherein A comprises a single chain variable fragment (scFv) that binds to CD3, wherein the scFv comprises a scFv light chain variable domain and a scFv heavy chain variable domain; B comprises an antigen binding fragment (Fab) or Fab′ that binds to EGFR, wherein the Fab or Fab′ comprises a Fab light chain polypeptide comprising a Fab light chain variable domain and a Fab heavy chain polypeptide comprising a Fab heavy chain variable domain comprising complementarity determining regions (CDRs): HC-CDR1, HC-CDR2, and HC-CDR3, wherein the HC-CDR1, the HC-CDR2, and the HC-CDR3 of the Fab heavy chain variable domain comprise HC-CDR1: SEQ ID NO: 18, HC-CDR2: SEQ ID NO: 19, and HC-CDR3: SEQ ID NO: 20, and wherein the Fab heavy chain polypeptide comprises an N-glycosylation site having an amino acid sequence of N-X₁-X₂, wherein X₁ is any amino acid except P and X₂ is T or S; and L comprises a linker that connects A to B, wherein the linker connects the Fab heavy chain polypeptide to the scFv light chain variable domain.

Disclosed herein, in some embodiments, are pharmaceutical compositions comprising an isolated polypeptide complex comprising the amino acid sequence of SEQ ID NO: 24 and the amino acid sequence of SEQ ID NO: 32.

Disclosed herein, in some embodiments, are pharmaceutical compositions comprising an isolated polypeptide complex comprising the amino acid sequence of SEQ ID NO: 26 and the amino acid sequence of SEQ ID NO: 32.

In some embodiments, the polypeptide or polypeptide complex further comprises a detectable label, a therapeutic agent, or a pharmacokinetic modifying moiety. In some embodiments, the detectable label comprises a fluorescent label, a radiolabel, an enzyme, a nucleic acid probe, or a contrast agent.

For administration to a subject, the polypeptide or polypeptide complex as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

### Isolated Recombinant Nucleic Acid Molecule

Disclosed herein are isolated recombinant nucleic acid molecules encoding an isolated polypeptide complex as disclosed herein. Described herein, in some embodiments, are isolated recombinant nucleic acid molecules encoding polypeptides comprising an antibody that selectively binds to CD3 and EGFR.

Disclosed herein, in some embodiments, are isolated recombinant nucleic acid molecules encoding an isolated polypeptide complex according to the formula:

(Formula I) A-L-B

wherein A comprises a single chain variable fragment (scFv) that binds to CD3, wherein the scFv comprises a scFv light chain variable domain and a scFv heavy chain variable domain comprising complementarity determining regions (CDRs): HC-CDR1, HC-CDR2, and HC-CDR3, wherein the HC-CDR1, the HC-CDR2, and the HC-CDR3 of the scFv heavy chain variable domain comprise HC-CDR1: SEQ ID NO: 1, HC-CDR2: SEQ ID NO: 2, and HC-CDR3: SEQ ID NO: 3; B comprises an antigen binding fragment (Fab) or Fab′ that binds to EGFR, wherein the Fab or Fab′ comprises a Fab light chain polypeptide comprising a Fab light chain variable domain and a Fab heavy chain polypeptide comprising a Fab heavy chain variable domain comprising complementarity determining regions (CDRs): HC-CDR1, HC-CDR2, and HC-CDR3, wherein the HC-CDR1, the HC-CDR2, and the HC-CDR3 of the Fab heavy chain variable domain comprise HC-CDR1: SEQ ID NO: 18, HC-CDR2: SEQ ID NO: 19, and HC-CDR3: SEQ ID NO: 20, and wherein the Fab heavy chain polypeptide comprises an N-glycosylation site having an amino acid sequence of N-X₁-X₂, wherein X₁ is any amino acid except P and X₂ is T or S; and L comprises a linker that connects A to B.

Disclosed herein, in some embodiments, are isolated recombinant nucleic acid molecules encoding isolated polypeptide complexes according to the formula:

A-L-B (Formula I)

wherein A comprises a single chain variable fragment (scFv) that binds to CD3, wherein the scFv comprises a scFv light chain variable domain and a scFv heavy chain variable domain; B comprises an antigen binding fragment (Fab) or Fab′ that binds to EGFR, wherein the Fab or Fab′ comprises a Fab light chain polypeptide comprising a Fab light chain variable domain and a Fab heavy chain polypeptide comprising a Fab heavy chain variable domain comprising complementarity determining regions (CDRs): HC-CDR1, HC-CDR2, and HC-CDR3, wherein the HC-CDR1, the HC-CDR2, and the HC-CDR3 of the Fab heavy chain variable domain comprise HC-CDR1: SEQ ID NO: 18, HC-CDR2: SEQ ID NO: 19, and HC-CDR3: SEQ ID NO: 20, and wherein the Fab heavy chain polypeptide comprises an N-glycosylation site having an amino acid sequence of N-X₁-X₂, wherein X₁ is any amino acid except P and X₂ is T or S; and L comprises a linker that connects A to B, wherein the linker connects the Fab heavy chain polypeptide to the scFv light chain variable domain.

Disclosed herein, in some embodiments, are isolated recombinant nucleic acid molecules encoding an isolated polypeptide complex comprising the amino acid sequence of SEQ ID NO: 24 and the amino acid sequence of SEQ ID NO: 32.

Disclosed herein, in some embodiments, are isolated recombinant nucleic acid molecules encoding an isolated polypeptide complex comprising the amino acid sequence of SEQ ID NO: 26 and the amino acid sequence of SEQ ID NO: 32.

### Methods of Treatment

Disclosed herein, in some embodiments, are methods of treating cancer in a subject in need thereof comprising administering to the subject the isolated polypeptide complex of any embodiment disclosed herein. In some embodiments, the cancer has cells that express EGFR. In some embodiments, the polypeptides or polypeptide complexes described herein are used in a method of treating renal cell carcinoma (RCC), colorectal cancer (CRC), squamous cell carcinoma of the head and Neck (SCCHN), non-small cell lung cancer (NSCLC), prostate cancer, breast cancer, colon/rectum cancer, head and neck cancer, esophagogastric cancer, liver cancer, glioblastoma, cervical cancer, ovarian cancer, bladder cancer, kidney cancer, or pancreatic cancer. In some embodiments, the polypeptides or polypeptide complexes described herein are used in a method of treating subjects who are resistant to EGFR inhibitor treatment. In some embodiments, the polypeptides or polypeptide complexes described herein are used in a method of treating subjects who harbor KRAS mutations. In some embodiments, the polypeptides or polypeptide complexes described herein are used in a method of treating subjects who are resistant to EGFR inhibitor treatment and harbor KRAS mutations.

Disclosed herein, in some embodiments, are methods of treating cancer in a subject in need thereof comprising administering to the subject an isolated polypeptide complex according to the following formula: A-L-B (Formula I) wherein A comprises a single chain variable fragment (scFv) that binds to CD3, wherein the scFv comprises a scFv light chain variable domain and a scFv heavy chain variable domain comprising complementarity determining regions (CDRs): HC-CDR1, HC-CDR2, and HC-CDR3, wherein the HC-CDR1, the HC-CDR2, and the HC-CDR3 of the scFv heavy chain variable domain comprise HC-CDR1: SEQ ID NO: 1, HC-CDR2: SEQ ID NO: 2, and HC-CDR3: SEQ ID NO: 3; B comprises an antigen binding fragment (Fab) or Fab′ that binds to EGFR, wherein the Fab or Fab′ comprises a Fab light chain polypeptide comprising a Fab light chain variable domain and a Fab heavy chain polypeptide comprising a Fab heavy chain variable domain comprising complementarity determining regions (CDRs): HC-CDR1, HC-CDR2, and HC-CDR3, wherein the HC-CDR1, the HC-CDR2, and the HC-CDR3 of the Fab heavy chain variable domain comprise HC-CDR1: SEQ ID NO: 18, HC-CDR2: SEQ ID NO: 19, and HC-CDR3: SEQ ID NO: 20, and wherein the Fab heavy chain polypeptide comprises an N-glycosylation site having an amino acid sequence of N-X₁-X₂, wherein X₁ is any amino acid except P and X₂ is T or S; and L comprises a linker that connects A to B.

Disclosed herein, in some embodiments, are methods of treating cancer in a subject in need thereof comprising administering to the subject an isolated polypeptide complex according to the following formula: A-L-B (Formula I) wherein A comprises a single chain variable fragment (scFv) that binds to CD3, wherein the scFv comprises a scFv light chain variable domain and a scFv heavy chain variable domain; B comprises an antigen binding fragment (Fab) or Fab′ that binds to EGFR, wherein the Fab or Fab′ comprises a Fab light chain polypeptide comprising a Fab light chain variable domain and a Fab heavy chain polypeptide comprising a Fab heavy chain variable domain comprising complementarity determining regions (CDRs): HC-CDR1, HC-CDR2, and HC-CDR3, wherein the HC-CDR1, the HC-CDR2, and the HC-CDR3 of the Fab heavy chain variable domain comprise HC-CDR1: SEQ ID NO: 18, HC-CDR2: SEQ ID NO: 19, and HC-CDR3: SEQ ID NO: 20, and wherein the Fab heavy chain polypeptide comprises an N-glycosylation site having an amino acid sequence of N-X₁-X₂, wherein X₁ is any amino acid except P and X₂ is T or S; and L comprises a linker that connects A to B, wherein the linker connects the Fab heavy chain polypeptide to the scFv light chain variable domain.

Production of Antibodies that Bind to EGFR and CD3

In some embodiments, polypeptides described herein are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11 (5): 155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

In some embodiments, vectors include any suitable vectors derived from either a eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichia pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or coli bacilli.

Exemplary prokaryotic host cells include, but are not limited to, BL21, Mach 1™, DH10B™, TOP10, DH5a, DH10Bac™, OmniMax™, MegaX™, DH12S™, INV110, TOP10F′, INVαF, TOP10/P3, ccdB Survival, PIR1, PIR2, Stb12™, Stb13™, or Stb14™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomyceses (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293 FT cell line, 293F cells, 293 H cells, CHO DG44 cells, CHO-S cells, CHO-K1 cells, FUT8 KO CHOK1, Expi293F™ cells, Flp-In™ T-REX™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REX™ Jurkat cell line, Per. C6 cells, T-REX™-293 cell line, T-REX™-CHO cell line, and T-REX™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary insect cell lines include, but are not limited to, strains from Chlamydomonas reinhardtii 137c, or Synechococcus elongatus PPC 7942.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a bispecific antibody comprising a first antigen-binding site that specifically binds to CD3 and a second antigen-binding site that specifically binds to EGFR as defined herein before.

The label or package insert indicates that the composition is used for treating the condition of choice. Moreover, the article of manufacture may comprise (a) a first container with a composition contained therein, wherein the composition comprises the bispecific antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EMBODIMENTS

Embodiment 1 comprises an isolated polypeptide complex according to the following formula: A-L-B (Formula I) wherein A comprises a single chain variable fragment (scFv) that binds to CD3, wherein the scFv comprises a scFv light chain variable domain and a scFv heavy chain variable domain comprising complementarity determining regions (CDRs): HC-CDR1, HC-CDR2, and HC-CDR3, wherein the HC-CDR1, the HC-CDR2, and the HC-CDR3 of the scFv heavy chain variable domain comprise HC-CDR1: SEQ ID NO: 1, HC-CDR2: SEQ ID NO: 2, and HC-CDR3: SEQ ID NO: 3; B comprises an antigen binding fragment (Fab) or Fab′ that binds to EGFR, wherein the Fab or Fab′ comprises a Fab light chain polypeptide comprising a Fab light chain variable domain and a Fab heavy chain polypeptide comprising a Fab heavy chain variable domain comprising complementarity determining regions (CDRs): HC-CDR1, HC-CDR2, and HC-CDR3, wherein the HC-CDR1, the HC-CDR2, and the HC-CDR3 of the Fab heavy chain variable domain comprise HC-CDR1: SEQ ID NO: 18, HC-CDR2: SEQ ID NO: 19, and HC-CDR3: SEQ ID NO: 20, and wherein the Fab heavy chain polypeptide comprises an N-glycosylation site having an amino acid sequence of N-X₁-X₂, wherein X₁ is any amino acid except P and X₂ is T or S; and L comprises a linker that connects A to B.

Embodiment 2 comprises the isolated polypeptide complex of embodiment 1, wherein the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise LC-CDR1: SEQ ID NO: 21, LC-CDR2: SEQ ID NO: 22, and LC-CDR3: SEQ ID NO: 23, and wherein the CDRs comprise from 0-2 amino acid modifications in at least one of the LC-CDR1, LC-CDR2, or LC-CDR3.

Embodiment 3 comprises the isolated polypeptide complex of embodiments 1 or 2, wherein the Fab light chain polypeptide comprises a mutation that eliminates pyroglutamic acid formation at an N-terminus of the Fab light chain polypeptide relative to a non-mutated form of the Fab light chain polypeptide.

Embodiment 4 comprises the isolated polypeptide complex of embodiment 3, wherein the mutation is a Q to X₃ mutation at residue number 1 of the of the N-terminus of the Fab light chain polypeptide, and wherein X₃ is an amino acid selected from the group consisting of: A, R, N, D, C, E, G, H, I, L, K, M, F, P, S, T, W, Y, or V.

Embodiment 5 comprises the isolated polypeptide complex of embodiment 4, wherein X₃ is selected from D and E.

Embodiment 6 comprises the isolated polypeptide complex of embodiment 5, wherein X₃ is D.

Embodiment 7 comprises the isolated polypeptide complex of embodiments 1 or 2, wherein the Fab light chain polypeptide comprises the amino acid sequence of SEQ ID NO: 24 or SEQ ID NO: 26.

Embodiment 8 comprises the isolated polypeptide complex of any one of embodiments 1 to 6, wherein the Fab light chain polypeptide comprises the amino acid sequence of SEQ ID NO: 26.

Embodiment 9 comprises the isolated polypeptide complex of embodiments 1 or 2, wherein the Fab light chain polypeptide comprises Q at residue number 1 of the N-terminus of the Fab light chain polypeptide.

Embodiment 10 comprises the isolated polypeptide complex of any one of embodiments 1, 2, or 9, wherein the Fab light chain polypeptide comprises the amino acid sequence of SEQ ID NO: 24.

Embodiment 11 comprises the isolated polypeptide complex of any one of the preceding embodiments, wherein X₂ is T.

Embodiment 12 comprises the isolated polypeptide complex of any one of the preceding embodiments, wherein X₁ is D.

Embodiment 13 comprises the isolated polypeptide complex of any one of the preceding embodiments, wherein the Fab heavy chain polypeptide that comprises the N-glycosylation site comprises the amino acid sequence QSNDTAIY (SEQ ID NO: 33).

Embodiment 14 comprises the isolated polypeptide complex of any one of the preceding embodiments, wherein the N of the N-glycosylation site is located at residue number 88 of the Fab heavy chain polypeptide.

Embodiment 15 comprises the isolated polypeptide complex of any one of the preceding embodiments, wherein the Fab heavy chain polypeptide comprises the amino acid sequence of SEQ ID NO: 25.

Embodiment 16 comprises the isolated polypeptide complex of embodiments 1 or 2, wherein the Fab light chain polypeptide comprises the amino acid sequence of SEQ ID NO: 24 and the Fab heavy chain polypeptide comprises the amino acid sequence of SEQ ID NO: 25.

Embodiment 17 comprises the isolated polypeptide complex of embodiments 1 or 2, wherein the Fab light chain polypeptide consists of the amino acid sequence of SEQ ID NO: 24 and the Fab heavy chain polypeptide consists of the amino acid sequence of SEQ ID NO: 25.

Embodiment 18 comprises the isolated polypeptide complex of any one of embodiments 1 to 6, wherein the Fab light chain polypeptide comprises the amino acid sequence of SEQ ID NO: 26 and the Fab heavy chain polypeptide comprises the amino acid sequence of SEQ ID NO: 25.

Embodiment 19 comprises the isolated polypeptide complex of any one of embodiments 1 to 6, wherein the Fab light chain polypeptide consists of the amino acid sequence of SEQ ID NO: 26 and the Fab heavy chain polypeptide consists of the amino acid sequence of SEQ ID NO: 25.

Embodiment 20 comprises the isolated polypeptide complex of any one of the preceding embodiments, wherein the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise LC-CDR1: SEQ ID NO: 6, LC-CDR2: SEQ ID NO: 7, and LC-CDR3: SEQ ID NO: 8.

Embodiment 21 comprises the isolated polypeptide complex of any one of the preceding embodiments, wherein the scFv heavy chain variable domain comprises an amino acid sequence that has at least 80% sequence identity to the amino acid sequence of SEQ ID NO: 13.

Embodiment 22 comprises the isolated polypeptide complex of any one of embodiments 1 to 20, wherein the scFv heavy chain variable domain comprises an amino acid sequence of at least 100 consecutive amino acid residues of SEQ ID NO: 13.

Embodiment 23 comprises the isolated polypeptide complex of any one of the preceding embodiments, wherein the scFv heavy chain variable domain comprises the amino acid sequence of SEQ ID NO: 13.

Embodiment 24 comprises the isolated polypeptide complex of any one of the preceding embodiments, wherein the scFv light chain variable domain comprises an amino acid sequence that has at least 80% sequence identity to the amino acid sequence of SEQ ID NO: 12.

Embodiment 25 comprises the isolated polypeptide complex of any one of embodiments 1 to 23, wherein the scFv light chain variable domain comprises an amino acid sequence of at least 100 consecutive amino acid residues of SEQ ID NO: 12.

Embodiment 26 comprises the isolated polypeptide complex of any one of the preceding embodiments, wherein the scFv light chain variable domain comprises the amino acid sequence of SEQ ID NO: 12.

Embodiment 27 comprises the isolated polypeptide complex of any one of the preceding embodiments, wherein the scFv comprises an amino acid sequence that has at least 80% sequence identity to the amino acid sequence of SEQ ID NO: 14.

Embodiment 28 comprises the isolated polypeptide complex of any one of embodiments 1 to 26, wherein the scFv comprises an amino acid sequence of at least 200 consecutive amino acid residues of SEQ ID NO: 14.

Embodiment 29 comprises the isolated polypeptide complex of any one of embodiments 1 to 26, wherein the scFv comprises an amino acid sequence of at least 225 consecutive amino acid residues of SEQ ID NO: 14.

Embodiment 30 comprises the isolated polypeptide complex of any one of the preceding embodiments, wherein the scFv comprises the amino acid sequence of SEQ ID NO: 14.

Embodiment 31 comprises the isolated polypeptide complex of any one of the preceding embodiments, wherein the linker connects the C-terminus of A to the N-terminus of B.

Embodiment 32 comprises the isolated polypeptide complex of any one of the preceding embodiments, wherein the linker connects the C-terminus of A to the N-terminus of the Fab light chain polypeptide.

Embodiment 33 comprises the isolated polypeptide complex of any one of embodiments 1 to 31, wherein the linker connects the C-terminus of A to the N-terminus of the Fab heavy chain polypeptide.

Embodiment 34 comprises the isolated polypeptide complex of any one of embodiments 1 to 31 and 33, wherein the linker connects the Fab heavy chain polypeptide to the C-terminus of the scFv light chain variable domain.

Embodiment 35 comprises the isolated polypeptide complex of any one of embodiments 1 to 30, wherein the linker connects the N-terminus of A to the C-terminus of B.

Embodiment 36 comprises the isolated polypeptide complex of any one of embodiments 1 to 30 and 35, wherein the linker connects the N-terminus of A to the C-terminus of the Fab heavy chain polypeptide.

Embodiment 37 comprises the isolated polypeptide complex of any one of embodiments 1 to 30 and 35, wherein the linker connects the N-terminus of A to the C-terminus of the Fab light chain polypeptide.

Embodiment 38 comprises the isolated polypeptide complex of any one of the preceding embodiments, wherein the linker is at least 5 amino acids in length.

Embodiment 39 comprises the isolated polypeptide complex of any one of the preceding embodiments, wherein the linker is no more than 10 amino acids in length.

Embodiment 40 comprises the isolated polypeptide complex of any one of the preceding embodiments, wherein the linker is no more than 30 amino acids in length.

Embodiment 41 comprises the isolated polypeptide complex of any one of the preceding embodiments, wherein the linker is at least 5 amino acids and no more than 30 amino acids in length.

Embodiment 42 comprises the isolated polypeptide complex of any one of the preceding embodiments, wherein the linker is 5 amino acids in length.

Embodiment 43 comprises the isolated polypeptide complex of any one of embodiments 1 to 37, wherein the linker comprises the amino acid sequence of SEQ ID NO: 28 (GGGGSGGGGSGGGGS), SEQ ID NO: 29 (GGGGS), or SEQ ID NO: 30 (GGGGSGGGS).

Embodiment 44 comprises the isolated polypeptide complex of embodiment 43, wherein the linker comprises the amino acid sequence of SEQ ID NO: 29 (GGGGS).

Embodiment 45 comprises the isolated polypeptide complex of any one of embodiments 1 to 31, 33-34, and 38 to 44, wherein the linker connects the Fab heavy chain polypeptide to the C-terminus of the scFv light chain variable domain, and wherein the Fab light chain polypeptide comprises an amino acid sequence that has at least 80% sequence identity to the amino acid sequence of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

Embodiment 46 comprises the isolated polypeptide complex of any one of embodiments 1 to 31, 33-34, and 38 to 44, wherein the linker connects the Fab heavy chain polypeptide to the C-terminus of the scFv light chain variable domain, and wherein the Fab light chain polypeptide comprises an amino acid sequence that has at least 90% sequence identity to the amino acid sequence of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

Embodiment 47 comprises the isolated polypeptide complex of any one of embodiments 1 to 31, 33-34, and 38 to 44, wherein the linker connects the Fab heavy chain polypeptide to the C-terminus of the scFv light chain variable domain, and wherein the Fab light chain polypeptide comprises an amino acid sequence of at least 200 consecutive amino acid residues of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

Embodiment 48 comprises the isolated polypeptide complex of any one of embodiments 1 to 31, 33-34, and 38 to 44, wherein the linker connects the Fab heavy chain polypeptide to the C-terminus of the scFv light chain variable domain, and wherein the Fab light chain polypeptide comprises an amino acid sequence according to SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

Embodiment 49 comprises the isolated polypeptide complex of any one of embodiments 1 to 2, 9 to 17, and 20-48, wherein the isolated polypeptide complex comprises an amino acid sequence with at least 90%, 95%, 96%, 97%, 98%, 99%, or 100% identity to SEQ ID NO: 24, and the amino acid sequence of SEQ ID NO: 32.

Embodiment 50 comprises the isolated polypeptide complex of any one of embodiments 1 to 2, 9 to 17, and 20-48, wherein the isolated polypeptide complex comprises amino acid sequences of SEQ ID NO: 24 and SEQ ID NO: 32.

Embodiment 51 comprises the isolated polypeptide complex of any one of embodiments 1 to 2, 9 to 17, and 20-48, wherein the isolated polypeptide complex consists of amino acid sequences of SEQ ID NO: 24 and SEQ ID NO: 32.

Embodiment 52 comprises the isolated polypeptide complex of any one of embodiments 1 to 8, 11 to 15, and 18 to 48, wherein the isolated polypeptide complex comprises an amino acid sequence with at least 90%, 95%, 96%, 97%, 98%, 99%, or 100% identity to SEQ ID NO: 26, and the amino acid sequence of SEQ ID NO: 32.

Embodiment 53 comprises the isolated polypeptide complex of any one of embodiments 1 to 8, 11 to 15, and 18 to 48, wherein the isolated polypeptide complex comprises amino acid sequences of SEQ ID NO: 26 and SEQ ID NO: 32.

Embodiment 54 comprises the isolated polypeptide complex of any one of embodiments 1 to 8, 11 to 15, and 18 to 48, wherein the isolated polypeptide complex consists of amino acid sequences of SEQ ID NO: 26 and SEQ ID NO: 32.

Embodiment 55 comprises an isolated polypeptide complex according to the following formula: A-L-B (Formula I) wherein A comprises a single chain variable fragment (scFv) that binds to CD3, wherein the scFv comprises a scFv light chain variable domain and a scFv heavy chain variable domain; B comprises an antigen binding fragment (Fab) or Fab′ that binds to EGFR, wherein the Fab or Fab′ comprises a Fab light chain polypeptide comprising a Fab light chain variable domain and a Fab heavy chain polypeptide comprising a Fab heavy chain variable domain comprising complementarity determining regions (CDRs): HC-CDR1, HC-CDR2, and HC-CDR3, wherein the HC-CDR1, the HC-CDR2, and the HC-CDR3 of the Fab heavy chain variable domain comprise HC-CDR1: SEQ ID NO: 18, HC-CDR2: SEQ ID NO: 19, and HC-CDR3: SEQ ID NO: 20, and wherein the Fab heavy chain polypeptide comprises an N-glycosylation site having an amino acid sequence of N-X₁-X₂, wherein X₁ is any amino acid except P and X₂ is T or S; and L comprises a linker that connects A to B, wherein the linker connects the Fab heavy chain polypeptide to the scFv light chain variable domain.

Embodiment 56 comprises the isolated polypeptide complex of embodiment 55, wherein the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise LC-CDR1: SEQ ID NO: 21, LC-CDR2: SEQ ID NO: 22, and LC-CDR3: SEQ ID NO: 23, and wherein the CDRs comprise from 0-2 amino acid modifications in at least one of the LC-CDR1, LC-CDR2, or LC-CDR3.

Embodiment 57 comprises the isolated polypeptide complex of embodiments 55 or 56, wherein the Fab light chain polypeptide comprises a mutation that eliminates pyroglutamic acid formation at an N-terminus of the Fab light chain polypeptide relative to a non-mutated form of the Fab light chain polypeptide.

Embodiment 58 comprises the isolated polypeptide complex of embodiment 57, wherein the mutation is a Q to X₃ mutation at residue number 1 of the of the N-terminus of the Fab light chain polypeptide, and wherein X₃ is an amino acid selected from the group consisting of: A, R, N, D, C, E, G, H, I, L, K, M, F, P, S, T, W, Y, or V.

Embodiment 59 comprises the isolated polypeptide complex of embodiment 58, wherein X₃ is selected from D and E.

Embodiment 60 comprises the isolated polypeptide complex of embodiment 58, wherein X₃ is D.

Embodiment 61 comprises the isolated polypeptide complex of any one of embodiments 55 to 60, wherein the Fab light chain polypeptide comprises the amino acid sequence of SEQ ID NO: 24 or SEQ ID NO: 26.

Embodiment 62 comprises the isolated polypeptide complex of any one of embodiments 55 to 61, wherein the Fab light chain polypeptide comprises the amino acid sequence of SEQ ID NO: 26.

Embodiment 63 comprises the isolated polypeptide complex of embodiments 55 or 56, wherein the Fab light chain polypeptide comprises Q at residue number 1 of the N-terminus of the Fab light chain polypeptide.

Embodiment 64 comprises the isolated polypeptide complex of any one of embodiments 55 to 56 and 63, wherein the Fab light chain polypeptide comprises the amino acid sequence of SEQ ID NO: 24.

Embodiment 65 comprises the isolated polypeptide complex of any one of embodiments 55 to 64, wherein X₂ is T.

Embodiment 66 comprises the isolated polypeptide complex of any one of embodiments 55 to 65, wherein X₁ is D.

Embodiment 67 comprises the isolated polypeptide complex of any one of embodiments 55 to 66, wherein the Fab heavy chain polypeptide that comprises the N-glycosylation site comprises an amino acid sequence of QSNDTAIY (SEQ ID NO: 33).

Embodiment 68 comprises the isolated polypeptide complex of any one of embodiments 55 to 67, wherein the N of the N-glycosylation site is located at residue number 88 of the Fab heavy chain polypeptide.

Embodiment 69 comprises the isolated polypeptide complex of any one of embodiments 55 to 68, wherein the Fab heavy chain polypeptide comprises the amino acid sequence of SEQ ID NO: 25.

Embodiment 70 comprises the isolated polypeptide complex of any one of embodiments 55 to 56, 61, and 63 to 69, wherein the Fab light chain polypeptide comprises the amino acid sequence of SEQ ID NO: 24 and the Fab heavy chain polypeptide comprises the amino acid sequence of SEQ ID NO: 25.

Embodiment 71 comprises the isolated polypeptide complex of any one of embodiments 55 to 56, 61, and 63 to 70, wherein the Fab light chain polypeptide consists of the amino acid sequence of SEQ ID NO: 24 and the Fab heavy chain polypeptide consists of the amino acid sequence of SEQ ID NO: 25.

Embodiment 72 comprises the isolated polypeptide complex of any one of embodiments 55 to 62 and 65 to 69, wherein the Fab light chain polypeptide comprises the amino acid sequence of SEQ ID NO: 26 and the Fab heavy chain polypeptide comprises the amino acid sequence of SEQ ID NO: 25.

Embodiment 73 comprises the isolated polypeptide complex of any one of embodiments 55 to 62 and 65 to 69, wherein the Fab light chain polypeptide consists of the amino acid sequence of SEQ ID NO: 26 and the Fab heavy chain polypeptide consists of the amino acid sequence of SEQ ID NO: 25.

Embodiment 74 comprises the isolated polypeptide complex of any one of embodiments 55 to 73, wherein the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise HC-CDR1: SEQ ID NO: 1, HC-CDR2: SEQ ID NO: 2, and HC-CDR3: SEQ ID NO: 3.

Embodiment 75 comprises the isolated polypeptide complex of any one of embodiments 55 to 74, wherein the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise LC-CDR1: SEQ ID NO: 6, LC-CDR2: SEQ ID NO: 7, and LC-CDR3: SEQ ID NO: 8.

Embodiment 76 comprises the isolated polypeptide complex of any one of embodiments 55 to 75, wherein the scFv heavy chain variable domain comprises an amino acid sequence that has at least 80% sequence identity to the amino acid sequence of SEQ ID NO: 13.

Embodiment 77 comprises the isolated polypeptide complex of any one of embodiments 55 to 75, wherein the scFv heavy chain variable domain comprises an amino acid sequence of at least 100 consecutive amino acid residues of SEQ ID NO: 13.

Embodiment 78 comprises the isolated polypeptide complex of any one of embodiments 55 to 77, wherein the scFv heavy chain variable domain comprises the amino acid sequence of SEQ ID NO: 13.

Embodiment 79 comprises the isolated polypeptide complex of any one of embodiments 55 to 78, wherein the scFv light chain variable domain comprises an amino acid sequence that has at least 80% sequence identity to the amino acid sequence of SEQ ID NO: 12.

Embodiment 80 comprises the isolated polypeptide complex of any one of embodiments 55 to 78, wherein the scFv light chain variable domain comprises an amino acid sequence of at least 100 consecutive amino acid residues of SEQ ID NO: 12.

Embodiment 81 comprises the isolated polypeptide complex of any one of embodiments 55 to 80, wherein the scFv light chain variable domain comprises the amino acid sequence of SEQ ID NO: 12.

Embodiment 82 comprises the isolated polypeptide complex of any one of embodiments 55 to 81, wherein the scFv comprises an amino acid sequence that has at least 80% sequence identity to the amino acid sequence of SEQ ID NO: 14.

Embodiment 83 comprises the isolated polypeptide complex of any one of embodiments 55 to 81, wherein the scFv comprises an amino acid sequence of at least 200 consecutive amino acid residues of SEQ ID NO: 14.

Embodiment 84 comprises the isolated polypeptide complex of any one of embodiments 55 to 81, wherein the scFv comprises an amino acid sequence of at least 225 consecutive amino acid residues of SEQ ID NO: 14.

Embodiment 85 comprises the isolated polypeptide complex of any one of embodiments 55 to 84, wherein the scFv comprises the amino acid sequence of SEQ ID NO: 14.

Embodiment 86 comprises the isolated polypeptide complex of any one of embodiments 55 to 85, wherein the linker connects the C-terminus of A to the N-terminus of B.

Embodiment 87 comprises the isolated polypeptide complex of any one of embodiments 55 to 86, wherein the linker connects the C-terminus of A to the N-terminus of the Fab heavy chain polypeptide.

Embodiment 88 comprises the isolated polypeptide complex of any one of embodiments 55 to 87, wherein the linker connects the Fab heavy chain polypeptide to the C-terminus of the scFv light chain variable domain.

Embodiment 89 comprises the isolated polypeptide complex of any one of embodiments 55 to 88, wherein the linker connects the N-terminus of the Fab heavy chain polypeptide to the C-terminus of the scFv light chain variable domain.

Embodiment 90 comprises the isolated polypeptide complex of any one of embodiments 55 to 85, wherein the linker connects the N-terminus of A to the C-terminus of B.

Embodiment 91 comprises the isolated polypeptide complex of any one of embodiments 55 to 90, wherein the linker is at least 5 amino acids in length.

Embodiment 92 comprises the isolated polypeptide complex of any one of embodiments 55 to 91, wherein the linker is no more than 10 amino acids in length.

Embodiment 93 comprises the isolated polypeptide complex of any one of embodiments 55 to 91, wherein the linker is no more than 30 amino acids in length.

Embodiment 94 comprises the isolated polypeptide complex of any one of embodiments 55 to 91, wherein the linker is at least 5 amino acids and no more than 30 amino acids in length.

Embodiments 95 comprises the isolated polypeptide complex of any one of embodiments 55 to 90, wherein the linker is 5 amino acids in length.

Embodiments 96 comprises the isolated polypeptide complex of any one of embodiments 55 to 90, wherein the linker comprises the amino acid sequence of SEQ ID NO: 28 (GGGGSGGGGSGGGGS), SEQ ID NO: 29 (GGGGS), or SEQ ID NO: 30 (GGGGSGGGS).

Embodiment 97 comprises the isolated polypeptide complex of embodiment 96, wherein the linker comprises the amino acid sequence of SEQ ID NO: 29 (GGGGS).

Embodiment 98 comprises the isolated polypeptide complex of any one of embodiments 55 to 89 and 91 to 97, wherein the linker connects the Fab heavy chain polypeptide to the C-terminus of the scFv light chain variable domain, and wherein the Fab light chain polypeptide comprises an amino acid sequence that has at least 80% sequence identity to the amino acid sequence of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

Embodiment 99 comprises the isolated polypeptide complex of any one of embodiments 55 to 89 and 91 to 97, wherein the linker connects the Fab heavy chain polypeptide to the C-terminus of the scFv light chain variable domain, and wherein the Fab light chain polypeptide comprises an amino acid sequence that has at least 90% sequence identity to the amino acid sequence of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

Embodiment 100 comprises the isolated polypeptide complex of any one of embodiments 55 to 89 and 91 to 97, wherein the linker connects the Fab heavy chain polypeptide to the C-terminus of the scFv light chain variable domain, and wherein the Fab light chain polypeptide comprises an amino acid sequence of at least 200 consecutive amino acid residues of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

Embodiment 101 comprises the isolated polypeptide complex of any one of embodiments 55 to 89 and 91 to 97, wherein the linker connects the Fab heavy chain polypeptide to the C-terminus of the scFv light chain variable domain, and wherein the Fab light chain polypeptide comprises an amino acid sequence according to SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

Embodiment 102 comprises the isolated polypeptide complex of any one of embodiments 55-56, 61, 63-71, and 74-101, wherein the isolated polypeptide complex comprises an amino acid sequence with at least 90%, 95%, 96%, 97%, 98%, 99%, or 100% identity to SEQ ID NO: 24, and the amino acid sequence of SEQ ID NO: 32.

Embodiment 103 comprises the isolated polypeptide complex of any one of embodiments 55-56, 61, 63-71, and 74-101, wherein the isolated polypeptide complex comprises the amino acid sequences of SEQ ID NO: 24 and SEQ ID NO: 32.

Embodiment 104 comprises the isolated polypeptide complex of any one of embodiments 55-56, 61, 63-71, and 74-101, wherein the isolated polypeptide complex consists of the amino acid sequences of SEQ ID NO: 24 and SEQ ID NO: 32.

Embodiment 105 comprises the isolated polypeptide complex of any one of embodiments 55-62, 65-69, and 72-101, wherein the isolated polypeptide complex comprises an amino acid sequence with at least 90%, 95%, 96%, 97%, 98%, 99%, or 100% identity to SEQ ID NO: 26, and the amino acid sequence of SEQ ID NO: 32.

Embodiment 106 comprises the isolated polypeptide complex of any one of embodiments 55-62, 65-69, and 72-101, wherein the isolated polypeptide complex comprises the amino acid sequences of SEQ ID NO: 26 and SEQ ID NO: 32.

Embodiment 107 comprises the isolated polypeptide complex of any one of embodiments 55-62, 65-69, and 72-101, wherein the isolated polypeptide complex consists of the amino acid sequences of SEQ ID NO: 26 and SEQ ID NO: 32.

Embodiment 108 comprises a pharmaceutical composition comprising: (i) the isolated polypeptide complex according to any one of embodiments 1 to 107, and (ii) a pharmaceutically acceptable excipient.

Embodiment 109 comprises an isolated recombinant nucleic acid molecule encoding an isolated polypeptide complex of any one of embodiments 1 to 107.

Embodiment 110 comprises a method of treating cancer in a subject in need thereof comprising administering to the subject the isolated polypeptide complex of any one of embodiments 1 to 107.

### EXAMPLES

### Example 1: EGFR Polypeptide Complex Binding to EGFR and CD3ε

The EGFR polypeptide complexes Ab-1, Ab-2, and Ab-3 were evaluated for EGFR and CD3ε binding. Ab-1, Ab-2, and Ab-3 comprise the sequences as listed in Table 8.

TABLE 8
Antibody Sequences that Bind to EGFR and CD3e

| | Amino Acid Sequence | |
|---|---|---|
| Construct Description | (N to C) | SEQ ID NO: |
| Ab-1 LC | QILLTQSPVILSVSPGERVSFSCRASQSIGTNIH | 24 |
| | WYQQRTNGSPRLLIKYASESISGIPSRFSGSGSG | |
| | TDFTLSINSVESEDIADYYCQQNNNWPTTFGA | |
| | GTKLELKRTVAAPSVFIFPPSDEQLKSGTASVV | |
| | CLLNNFYPREAKVQWKVDNALQSGNSQESVT | |
| | EQDSKDSTYSLSSTLTLSKADYEKHKVYACEV | |
| | THQGLSSPVTKSFNRGEC | |
| Ab-1 HC | EVQLVESGGGLVQPGGSLKLSCAASGFTFNKY | 31 |
| N-term SP34.184 scFv | AMNWVRQAPGKGLEWVARIRSKYNNYATYY | |
| | ADSVKDRFTISRDDSKNTAYLQMNNLKTEDTA | |
| | VYYCVRHGNFGNSYISYWAYWGQGTLVTVS | |
| | SGGGGSGGGGSGGGGSQTVVTQEPSLTVSPGG | |
| | TVTLTCGSSTGAVTSGNYPNWVQQKPGQAPR | |
| | GLIGGTKFLAPGTPARFSGSLLGGKAALTLSGV | |
| | QPEDEAEYYCVLWYSNRWVFGGGTKLTVLG | |
| | GGGSQVQLKQSGPGLVQPSQSLSITCTVSGFSL | |
| | TNYGVHWVRQSPGKGLEWLGVIWSGGNTDY | |
| | NTPFTSRLSINKDNSKSQVFFKMNSLQSQDTAI | |
| | YYCARALTYYDYEFAYWGQGTLVTVSAAST | |
| | KGPSVFPLAPSSKSTSGGTAALGCLVKDYFPEP | |
| | VTVSWNSGALTSGVHTFPAVLQSSGLYSLSSV | |
| | VTVPSSSLGTQTYICNVNHKPSNTKVDKKVEP | |
| | KSC | |
| Ab-2 LC | QILLTQSPVILSVSPGERVSFSCRASQSIGTNIH | 24 |
| | WYQQRTNGSPRLLIKYASESISGIPSRFSGSGSG | |
| | TDFTLSINSVESEDIADYYCQQNNNWPTTFGA | |
| | GTKLELKRTVAAPSVFIFPPSDEQLKSGTASVV | |
| | CLLNNFYPREAKVQWKVDNALQSGNSQESVT | |
| | EQDSKDSTYSLSSTLTLSKADYEKHKVYACEV | |
| | THQGLSSPVTKSFNRGEC | |
| Ab-2 HC | EVQLVESGGGLVQPGGSLKLSCAASGFTFNKY | 32 |
| N-term SP34 185 scFv | AMNWVRQAPGKGLEWVARIRSKYNNYATYY | |
| | ADSVKDRFTISRDDSKNTAYLQMNNLKTEDTA | |
| | VYYCVRHGNFGNSYISYWAYWGQGTLVTVS | |
| | SGGGGSGGGGSGGGGSQTVVTQEPSLTVSPGG | |
| | TVTLTCGSSTGAVTSGNYPNWVQQKPGQAPR | |
| | GLIGGTKFLAPGTPARFSGSLLGGKAALTLSGV | |
| | QPEDEAEYYCVLWYSNRWVFGGGTKLTVLG | |
| | GGGSQVQLKQSGPGLVQPSQSLSITCTVSGFSL | |
| | TNYGVHWVRQSPGKGLEWLGVIWSGGNTDY | |
| | NTPFTSRLSINKDNSKSQVFFKMNSLQSNDTAI | |
| | YYCARALTYYDYEFAYWGQGTLVTVSAAST | |
| | KGPSVFPLAPSSKSTSGGTAALGCLVKDYFPEP | |
| | VTVSWNSGALTSGVHTFPAVLQSSGLYSLSSV | |
| | VTVPSSSLGTQTYICNVNHKPSNTKVDKKVEP | |
| | KSC | |
| Ab-3 LC | DILLTQSPVILSVSPGERVSFSCRASQSIGTNIH | 26 |
| | WYQQRTNGSPRLLIKYASESISGIPSRFSGSGSG | |
| | TDFTLSINSVESEDIADYYCQQNNNWPTTFGA | |
| | GTKLELKRTVAAPSVFIFPPSDEQLKSGTASVV | |
| | CLLNNFYPREAKVQWKVDNALQSGNSQESVT | |
| | EQDSKDSTYSLSSTLTLSKADYEKHKVYACEV | |
| | THQGLSSPVTKSFNRGEC | |
| Ab-3 HC | EVQLVESGGGLVQPGGSLKLSCAASGFTFNKY | 32 |
| N-term SP34.185 scFv | AMNWVRQAPGKGLEWVARIRSKYNNYATYY | |
| | ADSVKDRFTISRDDSKNTAYLQMNNLKTEDTA | |
| | VYYCVRHGNFGNSYISYWAYWGQGTLVTVS | |
| | SGGGGSGGGGSGGGGSQTVVTQEPSLTVSPGG | |
| | TVTLTCGSSTGAVTSGNYPNWVQQKPGQAPR | |
| | GLIGGTKFLAPGTPARFSGSLLGGKAALTLSGV | |
| | QPEDEAEYYCVLWYSNRWVFGGGTKLTVLG | |
| | GGGSQVQLKQSGPGLVQPSQSLSITCTVSGFSL | |
| | TNYGVHWVRQSPGKGLEWLGVIWSGGNTDY | |
| | NTPFTSRLSINKDNSKSQVFFKMNSLQSNDTAI | |
| | YYCARALTYYDYEFAYWGQGTLVTVSAAST | |
| | KGPSVFPLAPSSKSTSGGTAALGCLVKDYFPEP | |
| | VTVSWNSGALTSGVHTFPAVLQSSGLYSLSSV | |
| | VTVPSSSLGTQTYICNVNHKPSNTKVDKKVEP | |
| | KSC | |

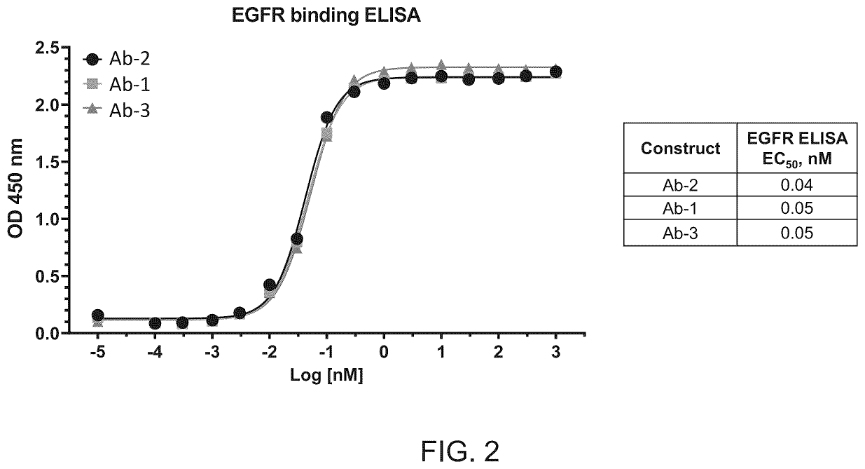

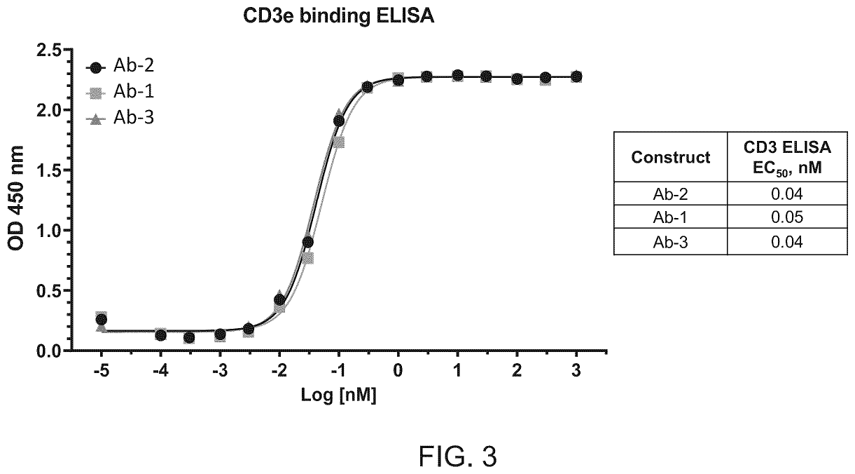

Ab-1, Ab-2, and Ab-3 binding to EGFR and CD3 was evaluated using enzyme linked immunosorbent assays (ELISAs). Biotinylated EGFR or biotinylated CD3 were captured on neutravidin coated plates. Polypeptide complex molecules diluted in buffer were added to the antigen coated plates. Bound polypeptide complex was detected using a standard horseradish peroxidase conjugated secondary antibody. The concentration of polypeptide complex required to achieve 50% maximal signal (EC₅₀) was calculated using Graphpad Prism software. Binding data is seen in FIG. 2 for Ab-1, Ab-2, and Ab-3 binding to EGFR. Binding data is seen in FIG. 3 for Ab-1, Ab-2, and Ab-3 binding to CD3. EC₅₀s for EGFR and CD3 binding by all three polypeptides are shown in FIGS. 2-3 and in Table 9.

TABLE 9
EC₅₀s for EGFR and CD3 Binding

EC₅₀, nM

| Polypeptide | EGFR | CD3 |
|---|---|---|
| Ab-1 | 0.05 | 0.05 |
| Ab-2 | 0.04 | 0.04 |
| Ab-3 | 0.05 | 0.04 |

### Example 2: In Vitro Efficacy of EGFR Polypeptide Complexes

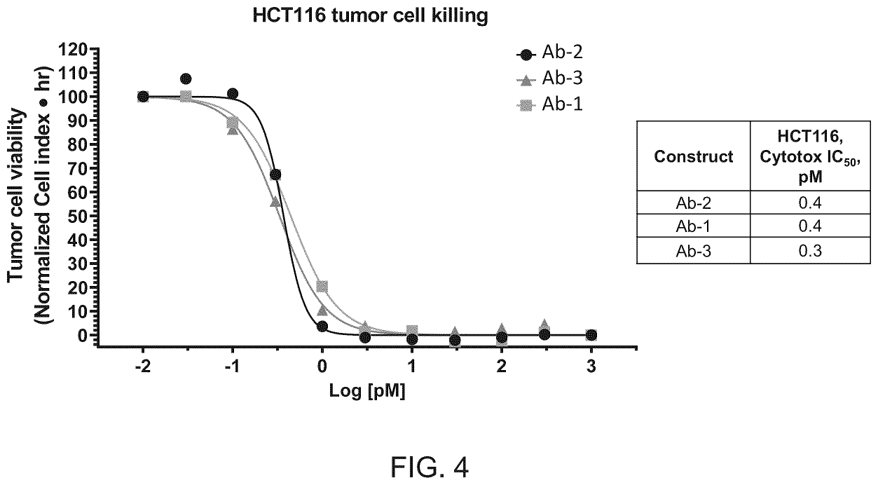

Polypeptide complexes were evaluated in a functional in vitro tumor cell killing assay using the EGFR positive tumor cell lines HCT116. Tumor cell killing was measured using an xCelligence real time cell analyzer from Agilent that relies on sensor impedance measurements (cell index) that increased as tumor cells adhere, spread, and expand on the surface of the sensor. Likewise, as the tumor cells were killed the impedance decreased. 10,000 tumor cells were added per well and allowed to adhere overnight on a 96 well E-Plate. The following day, polypeptide complexes titrated in human serum supplemented medium along with 30,000 CD8+ T cells were added to the wells. Cell index measurements were taken every 10 minutes for an additional 72 hours. The cell index times number of hours (tumor cell growth kinetics) was then plotted versus concentration of polypeptide complex. The concentration required to reduce the tumor growth 50% (IC₅₀) was calculated using Graphpad Prism software. Tumor cell killing plots with Ab-1, Ab-2, and Ab-3 are shown in FIG. 4, and IC₅₀s are provided in FIG. 4 and Table 10.

TABLE 10
IC₅₀s for HCT116 Tumor Cell Killing

| Polypeptide | IC₅₀ (pM) |
|---|---|
| Ab-1 | 0.4 |
| Ab-2 | 0.4 |
| Ab-3 | 0.3 |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby

What is claimed is:

1. An isolated polypeptide complex according to the following formula:
A-L-B (Formula I)
wherein
A comprises a single chain variable fragment (scFv) that binds to CD3, wherein the scFv comprises a scFv light chain variable domain and a scFv heavy chain variable domain comprising complementarity determining regions (CDRs): HC-CDR1, HC-CDR2, and HC-CDR3, wherein the HC-CDR1, the HC-CDR2, and the HC-CDR3 of the scFv heavy chain variable domain comprise HC-CDR1: SEQ ID NO: 1, HC-CDR2: SEQ ID NO: 2, and HC-CDR3: SEQ ID NO: 3;
B comprises an antigen binding fragment (Fab) or Fab′ that binds to EGFR, wherein the Fab or Fab′ comprises a Fab light chain polypeptide comprising a Fab light chain variable domain and a Fab heavy chain polypeptide comprising a Fab heavy chain variable domain comprising complementarity determining regions (CDRs): HC-CDR1, HC-CDR2, and HC-CDR3, wherein the HC-CDR1, the HC-CDR2, and the HC-CDR3 of the Fab heavy chain variable domain comprise HC-CDR1: SEQ ID NO: 18, HC-CDR2: SEQ ID NO: 19, and HC-CDR3: SEQ ID NO: 20, and wherein the Fab heavy chain polypeptide comprises an N-glycosylation site having an amino acid sequence of N-X₁-X₂, wherein X₁ is any amino acid except P and X₂ is T or S; and
L comprises a linker that connects A to B.

2. The isolated polypeptide complex of claim 1, wherein the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise LC-CDR1: SEQ ID NO: 21, LC-CDR2: SEQ ID NO: 22, and LC-CDR3: SEQ ID NO: 23, and wherein the CDRs comprise from 0-2 amino acid modifications in at least one of the LC-CDR1, LC-CDR2, or LC-CDR3.

3. The isolated polypeptide complex of claim 1, wherein the Fab light chain polypeptide comprises a mutation that eliminates pyroglutamic acid formation at an N-terminus of the Fab light chain polypeptide relative to a non-mutated form of the Fab light chain polypeptide.

4. The isolated polypeptide complex of claim 3, wherein the mutation is a Q to X₃ mutation at residue number 1 of the of the N-terminus of the Fab light chain polypeptide, and wherein X₃ is an amino acid selected from the group consisting of: A, R, N, D, C, E, G, H, I, L, K, M, F, P, S, T, W, Y, or V.

5. The isolated polypeptide complex of claim 4, wherein X₃ is selected from D and E.

6. The isolated polypeptide complex of claim 5, wherein X₃ is D.

7. The isolated polypeptide complex of claim 1, wherein the Fab light chain polypeptide comprises the amino acid sequence of SEQ ID NO: 24 or SEQ ID NO: 26.

8. The isolated polypeptide complex of claim 1, wherein the Fab light chain polypeptide comprises the amino acid sequence of SEQ ID NO: 26.

9. The isolated polypeptide complex of claim 1, wherein the Fab light chain polypeptide comprises Q at residue number 1 of the N-terminus of the Fab light chain polypeptide.

10. The isolated polypeptide complex of claim 1, wherein the Fab light chain polypeptide comprises the amino acid sequence of SEQ ID NO: 24.

11. The isolated polypeptide complex of claim 1, wherein X₂ is T.

12. The isolated polypeptide complex of claim 11, wherein X₁ is D.

13. The isolated polypeptide complex of claim 1, wherein the Fab heavy chain polypeptide that comprises the N-glycosylation site comprises the amino acid sequence QSNDTAIY (SEQ ID NO: 33).

14. The isolated polypeptide complex of claim 1, wherein the N of the N-glycosylation site is located at residue number 88 of the Fab heavy chain polypeptide.

15. The isolated polypeptide complex of claim 1, wherein the Fab heavy chain polypeptide comprises the amino acid sequence of SEQ ID NO: 25.

16. The isolated polypeptide complex of claim 1, wherein the Fab light chain polypeptide comprises the amino acid sequence of SEQ ID NO: 24 and the Fab heavy chain polypeptide comprises the amino acid sequence of SEQ ID NO: 25.

17. The isolated polypeptide complex of claim 1, wherein the Fab light chain polypeptide consists of the amino acid sequence of SEQ ID NO: 24 and the Fab heavy chain polypeptide consists of the amino acid sequence of SEQ ID NO: 25.

18. The isolated polypeptide complex of claim 1, wherein the Fab light chain polypeptide comprises the amino acid sequence of SEQ ID NO: 26 and the Fab heavy chain polypeptide comprises the amino acid sequence of SEQ ID NO: 25.

19. The isolated polypeptide complex of claim 1, wherein the Fab light chain polypeptide consists of the amino acid sequence of SEQ ID NO: 26 and the Fab heavy chain polypeptide consists of the amino acid sequence of SEQ ID NO: 25.

20. The isolated polypeptide complex of claim 1, wherein the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise LC-CDR1: SEQ ID NO: 6, LC-CDR2: SEQ ID NO: 7, and LC-CDR3: SEQ ID NO: 8.

21. The isolated polypeptide complex of claim 1, wherein the scFv heavy chain variable domain comprises an amino acid sequence that has at least 80% sequence identity to the amino acid sequence of SEQ ID NO: 13.

22. The isolated polypeptide complex of claim 1, wherein the scFv heavy chain variable domain comprises an amino acid sequence of at least 100 consecutive amino acid residues of SEQ ID NO: 13.

23. The isolated polypeptide complex of claim 1, wherein the scFv heavy chain variable domain comprises the amino acid sequence of SEQ ID NO: 13.

24. The isolated polypeptide complex of claim 1, wherein the scFv light chain variable domain comprises an amino acid sequence that has at least 80% sequence identity to the amino acid sequence of SEQ ID NO: 12.

25. The isolated polypeptide complex of claim 1, wherein the scFv light chain variable domain comprises an amino acid sequence of at least 100 consecutive amino acid residues of SEQ ID NO: 12.

26. The isolated polypeptide complex of claim 1, wherein the scFv light chain variable domain comprises the amino acid sequence of SEQ ID NO: 12.

27. The isolated polypeptide complex of claim 1, wherein the scFv comprises an amino acid sequence that has at least 80% sequence identity to the amino acid sequence of SEQ ID NO: 14.

28. The isolated polypeptide complex of claim 1, wherein the scFv comprises an amino acid sequence of at least 200 consecutive amino acid residues of SEQ ID NO: 14.

29. The isolated polypeptide complex of claim 1, wherein the scFv comprises an amino acid sequence of at least 225 consecutive amino acid residues of SEQ ID NO: 14.

30. The isolated polypeptide complex of claim 1, wherein the scFv comprises the amino acid sequence of SEQ ID NO: 14.

31. The isolated polypeptide complex of claim 1, wherein the linker connects the C-terminus of A to the N-terminus of B.

32. The isolated polypeptide complex of claim 1, wherein the linker connects the C-terminus of A to the N-terminus of the Fab light chain polypeptide.

33. The isolated polypeptide complex of claim 1, wherein the linker connects the C-terminus of A to the N-terminus of the Fab heavy chain polypeptide.

34. The isolated polypeptide complex of claim 1, wherein the linker connects the Fab heavy chain polypeptide to the C-terminus of the scFv light chain variable domain.

35. The isolated polypeptide complex of claim 1, wherein the linker connects the N-terminus of A to the C-terminus of B.

36. The isolated polypeptide complex of claim 1, wherein the linker connects the N-terminus of A to the C-terminus of the Fab heavy chain polypeptide.

37. The isolated polypeptide complex of claim 1, wherein the linker connects the N-terminus of A to the C-terminus of the Fab light chain polypeptide.

38. The isolated polypeptide complex of claim 1, wherein the linker is at least 5 amino acids in length.

39. The isolated polypeptide complex of claim 1, wherein the linker is no more than 10 amino acids in length.

40. The isolated polypeptide complex of claim 1, wherein the linker is no more than 30 amino acids in length.

41. The isolated polypeptide complex of claim 1, wherein the linker is at least 5 amino acids and no more than 30 amino acids in length.

42. The isolated polypeptide complex of claim 1, wherein the linker is 5 amino acids in length.

43. The isolated polypeptide complex of claim 1, wherein the linker comprises the amino acid sequence of SEQ ID NO: 28 (GGGGSGGGGSGGGGS), SEQ ID NO: 29 (GGGGS), or SEQ ID NO: 30 (GGGGSGGGS).

44. The isolated polypeptide complex of claim 1, wherein the linker comprises the amino acid sequence of SEQ ID NO: 29 (GGGGS).

45. The isolated polypeptide complex of claim 1, wherein the linker connects the Fab heavy chain polypeptide to the C-terminus of the scFv light chain variable domain, and wherein the Fab light chain polypeptide comprises an amino acid sequence that has at least 80% sequence identity to the amino acid sequence of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

46. The isolated polypeptide complex of claim 1, wherein the linker connects the Fab heavy chain polypeptide to the C-terminus of the scFv light chain variable domain, and wherein the Fab light chain polypeptide comprises an amino acid sequence that has at least 90% sequence identity to the amino acid sequence of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

47. The isolated polypeptide complex of claim 1, wherein the linker connects the Fab heavy chain polypeptide to the C-terminus of the scFv light chain variable domain, and wherein the Fab light chain polypeptide comprises an amino acid sequence of at least 200 consecutive amino acid residues of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

48. The isolated polypeptide complex of claim 1, wherein the linker connects the Fab heavy chain polypeptide to the C-terminus of the scFv light chain variable domain, and wherein the Fab light chain polypeptide comprises an amino acid sequence according to SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

49. The isolated polypeptide complex of claim 1, wherein the isolated polypeptide complex comprises an amino acid sequence with at least 90%, 95%, 96%, 97%, 98%, 99%, or 100% identity to SEQ ID NO: 24, and the amino acid sequence of SEQ ID NO: 32.

50. The isolated polypeptide complex of claim 1, wherein the isolated polypeptide complex comprises the amino acid sequences of SEQ ID NO: 24 and SEQ ID NO: 32.

51. The isolated polypeptide complex of claim 1, wherein the isolated polypeptide complex consists of the amino acid sequences of SEQ ID NO: 24 and SEQ ID NO: 32.

52. The isolated polypeptide complex of claim 1, wherein the isolated polypeptide complex comprises an amino acid sequence with at least 90%, 95%, 96%, 97%, 98%, 99%, or 100% identity to SEQ ID NO: 26, and the amino acid sequence of SEQ ID NO: 32.

53. The isolated polypeptide complex of claim 1, wherein the isolated polypeptide complex comprises the amino acid sequences of SEQ ID NO: 26 and SEQ ID NO: 32.

54. The isolated polypeptide complex of claim 1, wherein the isolated polypeptide complex consists of the amino acid sequences of SEQ ID NO: 26 and SEQ ID NO: 32.

55. An isolated polypeptide complex according to the following formula:
A-L-B (Formula I)
wherein
A comprises a single chain variable fragment (scFv) that binds to CD3, wherein the scFv comprises a scFv light chain variable domain and a scFv heavy chain variable domain;
B comprises an antigen binding fragment (Fab) or Fab′ that binds to EGFR, wherein the Fab or Fab′ comprises a Fab light chain polypeptide comprising a Fab light chain variable domain and a Fab heavy chain polypeptide comprising a Fab heavy chain variable domain comprising complementarity determining regions (CDRs): HC-CDR1, HC-CDR2, and HC-CDR3, wherein the HC-CDR1, the HC-CDR2, and the HC-CDR3 of the Fab heavy chain variable domain comprise HC-CDR1: SEQ ID NO: 18, HC-CDR2: SEQ ID NO: 19, and HC-CDR3: SEQ ID NO: 20, and wherein the Fab heavy chain polypeptide comprises an N-glycosylation site having an amino acid sequence of N-X₁-X₂, wherein X₁ is any amino acid except P and X₂ is T or S; and
L comprises a linker that connects A to B, wherein the linker connects the Fab heavy chain polypeptide to the scFv light chain variable domain.

56. The isolated polypeptide complex of claim 55, wherein the Fab light chain variable domain comprises complementarity determining regions (CDRs): LC-CDR1, LC-CDR2, and LC-CDR3, wherein the LC-CDR1, the LC-CDR2, and the LC-CDR3 of the Fab light chain variable domain comprise LC-CDR1: SEQ ID NO: 21, LC-CDR2: SEQ ID NO: 22, and LC-CDR3: SEQ ID NO: 23, and wherein the CDRs comprise from 0-2 amino acid modifications in at least one of the LC-CDR1, LC-CDR2, or LC-CDR3.

57. The isolated polypeptide complex of claim 55, wherein the Fab light chain polypeptide comprises a mutation that eliminates pyroglutamic acid formation at an N-terminus of the Fab light chain polypeptide relative to a non-mutated form of the Fab light chain polypeptide.

58. The isolated polypeptide complex of claim 57, wherein the mutation is a Q to X₃ mutation at residue number 1 of the of the N-terminus of the Fab light chain polypeptide, and wherein X₃ is an amino acid selected from the group consisting of: A, R, N, D, C, E, G, H, I, L, K, M, F, P, S, T, W, Y, or V.

59. The isolated polypeptide complex of claim 58, wherein X₃ is selected from D and E.

60. The isolated polypeptide complex of claim 58, wherein X₃ is D.

61. The isolated polypeptide complex of claim 55, wherein the Fab light chain polypeptide comprises the amino acid sequence of SEQ ID NO: 24 or SEQ ID NO: 26.

62. The isolated polypeptide complex of claim 55, wherein the Fab light chain polypeptide comprises the amino acid sequence of SEQ ID NO: 26.

63. The isolated polypeptide complex of claim 55, wherein the Fab light chain polypeptide comprises Q at residue number 1 of the N-terminus of the Fab light chain polypeptide.

64. The isolated polypeptide complex of claim 55, wherein the Fab light chain polypeptide comprises the amino acid sequence of SEQ ID NO: 24.

65. The isolated polypeptide complex of claim 55, wherein X₂ is T.

66. The isolated polypeptide complex of claim 65, wherein X₁ is D.

67. The isolated polypeptide complex of claim 55, wherein the Fab heavy chain polypeptide that comprises the N-glycosylation site comprises an amino acid sequence of QSNDTAIY (SEQ ID NO: 33).

68. The isolated polypeptide complex of claim 55, wherein the N of the N-glycosylation site is located at residue number 88 of the Fab heavy chain polypeptide.

69. The isolated polypeptide complex of claim 55, wherein the Fab heavy chain polypeptide comprises the amino acid sequence of SEQ ID NO: 25.

70. The isolated polypeptide complex of claim 55, wherein the Fab light chain polypeptide comprises the amino acid sequence of SEQ ID NO: 24 and the Fab heavy chain polypeptide comprises the amino acid sequence of SEQ ID NO: 25.

71. The isolated polypeptide complex of claim 55, wherein the Fab light chain polypeptide consists of the amino acid sequence of SEQ ID NO: 24 and the Fab heavy chain polypeptide consists of the amino acid sequence of SEQ ID NO: 25.

72. The isolated polypeptide complex of claim 55, wherein the Fab light chain polypeptide comprises the amino acid sequence of SEQ ID NO: 26 and the Fab heavy chain polypeptide comprises the amino acid sequence of SEQ ID NO: 25.

73. The isolated polypeptide complex of claim 55, wherein the Fab light chain polypeptide consists of the amino acid sequence of SEQ ID NO: 26 and the Fab heavy chain polypeptide consists of the amino acid sequence of SEQ ID NO: 25.

74. The isolated polypeptide complex of claim 55, wherein the scFv heavy chain variable domain comprises complementarity determining regions (CDRs): HC-CDR1, HC-CDR2, and HC-CDR3, wherein the HC-CDR1, the HC-CDR2, and the HC-CDR3 of the scFv heavy chain variable domain comprise HC-CDR1: SEQ ID NO: 1, HC-CDR2: SEQ ID NO: 2, and HC-CDR3: SEQ ID NO: 3.

75. The isolated polypeptide complex of claim 55, wherein the scFv light chain variable domain comprises complementarity determining regions (CDRs): LC-CDR1, LC-CDR2, and LC-CDR3, wherein the LC-CDR1, the LC-CDR2, and the LC-CDR3 of the scFv light chain variable domain comprise LC-CDR1: SEQ ID NO: 6, LC-CDR2: SEQ ID NO: 7, and LC-CDR3: SEQ ID NO: 8.

76. The isolated polypeptide complex of claim 55, wherein the scFv heavy chain variable domain comprises an amino acid sequence that has at least 80% sequence identity to the amino acid sequence of SEQ ID NO: 13.

77. The isolated polypeptide complex of claim 55, wherein the scFv heavy chain variable domain comprises an amino acid sequence of at least 100 consecutive amino acid residues of SEQ ID NO: 13.

78. The isolated polypeptide complex of claim 55, wherein the scFv heavy chain variable domain comprises the amino acid sequence of SEQ ID NO: 13.

79. The isolated polypeptide complex of claim 55, wherein the scFv light chain variable domain comprises an amino acid sequence that has at least 80% sequence identity to the amino acid sequence of SEQ ID NO: 12.

80. The isolated polypeptide complex of claim 55, wherein the scFv light chain variable domain comprises an amino acid sequence of at least 100 consecutive amino acid residues of SEQ ID NO: 12.

81. The isolated polypeptide complex of claim 55, wherein the scFv light chain variable domain comprises the amino acid sequence of SEQ ID NO: 12.

82. The isolated polypeptide complex of claim 55, wherein the scFv comprises an amino acid sequence that has at least 80% sequence identity to the amino acid sequence of SEQ ID NO: 14.

83. The isolated polypeptide complex of claim 55, wherein the scFv comprises an amino acid sequence of at least 200 consecutive amino acid residues of SEQ ID NO: 14.

84. The isolated polypeptide complex of claim 55, wherein the scFv comprises an amino acid sequence of at least 225 consecutive amino acid residues of SEQ ID NO: 14.

85. The isolated polypeptide complex of claim 55, wherein the scFv comprises the amino acid sequence of SEQ ID NO: 14.

86. The isolated polypeptide complex of claim 55, wherein the linker connects the C-terminus of A to the N-terminus of B.

87. The isolated polypeptide complex of claim 55, wherein the linker connects the C-terminus of A to the N-terminus of the Fab heavy chain polypeptide.

88. The isolated polypeptide complex of claim 55, wherein the linker connects the Fab heavy chain polypeptide to the C-terminus of the scFv light chain variable domain.

89. The isolated polypeptide complex of claim 55, wherein the linker connects the N-terminus of the Fab heavy chain polypeptide to the C-terminus of the scFv light chain variable domain.

90. The isolated polypeptide complex of claim 55, wherein the linker connects the N-terminus of A to the C-terminus of B.

91. The isolated polypeptide complex of claim 55, wherein the linker is at least 5 amino acids in length.

92. The isolated polypeptide complex of claim 55, wherein the linker is no more than 10 amino acids in length.

93. The isolated polypeptide complex of claim 55, wherein the linker is no more than 30 amino acids in length.

94. The isolated polypeptide complex of claim 55, wherein the linker is at least 5 amino acids and no more than 30 amino acids in length.

95. The isolated polypeptide complex of claim 55, wherein the linker is 5 amino acids in length.

96. The isolated polypeptide complex of claim 55, wherein the linker comprises the amino acid sequence of SEQ ID NO: 28 (GGGGSGGGGSGGGGS), SEQ ID NO: 29 (GGGGS), or SEQ ID NO: 30 (GGGGSGGGS).

97. The isolated polypeptide complex of claim 55, wherein the linker comprises the amino acid sequence of SEQ ID NO: 29 (GGGGS).

98. The isolated polypeptide complex of claim 55, wherein the linker connects the Fab heavy chain polypeptide to the C-terminus of the scFv light chain variable domain, and wherein the Fab light chain polypeptide comprises an amino acid sequence that has at least 80% sequence identity to the amino acid sequence of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

99. The isolated polypeptide complex of claim 55, wherein the linker connects the Fab heavy chain polypeptide to the C-terminus of the scFv light chain variable domain, and wherein the Fab light chain polypeptide comprises an amino acid sequence that has at least 90% sequence identity to the amino acid sequence of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

100. The isolated polypeptide complex of claim 55, wherein the linker connects the Fab heavy chain polypeptide to the C-terminus of the scFv light chain variable domain, and wherein the Fab light chain polypeptide comprises an amino acid sequence of at least 200 consecutive amino acid residues of SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

101. The isolated polypeptide complex of claim 55, wherein the linker connects the Fab heavy chain polypeptide to the C-terminus of the scFv light chain variable domain, and wherein the Fab light chain polypeptide comprises an amino acid sequence according to SEQ ID NO: 24 or SEQ ID NO: 26, and an amino acid sequence of the Fab heavy chain polypeptide that is connected to the C-terminus of the scFv light chain variable domain comprises the amino acid sequence of SEQ ID NO: 32.

102. A pharmaceutical composition comprising:
(i) the isolated polypeptide complex of claim 1; and
(ii) a pharmaceutically acceptable excipient.

103. An isolated recombinant nucleic acid molecule encoding an isolated polypeptide complex of claim 1.

104. A method of treating cancer in a subject in need thereof comprising administering to the subject the isolated polypeptide complex of claim 1.


SEQUENCE LISTING (33 sequences)

SEQ ID NO: 1 - protein, 8 aa, synthetic construct
GFTFNKYA

SEQ ID NO: 2 - protein, 10 aa, synthetic construct
IRSKYNNYAT

SEQ ID NO: 3 - protein, 16 aa, synthetic construct
VRHGNFGNSYISYWAY

SEQ ID NO: 4 - protein, 8 aa, synthetic construct
GFTFNTYA

SEQ ID NO: 5 - protein, 16 aa, synthetic construct
VRHGNFGNSYVSWFAY

SEQ ID NO: 6 - protein, 9 aa, synthetic construct
TGAVTSGNY

SEQ ID NO: 7 - skipped

SEQ ID NO: 8 - protein, 9 aa, synthetic construct
VLWYSNRWV

SEQ ID NO: 9 - protein, 9 aa, synthetic construct
TGAVTTSNY

SEQ ID NO: 10 - skipped

SEQ ID NO: 11 - protein, 9 aa, synthetic construct
ALWYSNLWV

SEQ ID NO: 12 - protein, 109 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 13 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 14 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 15 - protein, 109 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 16 - protein, 125 aa, synthetic construct
EVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 17 - protein, 249 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 18 - protein, 8 aa, synthetic construct
GFSLTNYG

SEQ ID NO: 19 - protein, 7 aa, synthetic construct
IWSGGNT

SEQ ID NO: 20 - protein, 13 aa, synthetic construct
ARALTYYDYEFAY

SEQ ID NO: 21 - protein, 6 aa, synthetic construct
QSIGTN

SEQ ID NO: 22 - skipped

SEQ ID NO: 23 - protein, 9 aa, synthetic construct
QQNNNWPTT

SEQ ID NO: 24 - protein, 214 aa, synthetic construct
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 222 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 26 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 222 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 28 - protein, 15 aa, synthetic construct
GGGGSGGGGSGGGGS

SEQ ID NO: 29 - protein, 5 aa, synthetic construct
GGGGS

SEQ ID NO: 30 - protein, 9 aa, synthetic construct
GGGGSGGGS

SEQ ID NO: 31 - protein, 476 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 32 - protein, 476 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 33 - protein, 8 aa, synthetic construct
QSNDTAIY